SEQUENCE LISTING (12 sequences)

SEQ ID NO: 1 - DNA, 617 bp, Homo sapiens
gctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacactatgttacatactctaatatagtagtgaaagtcatttctttgtattccaagtggaggag

SEQ ID NO: 2 - protein, 131 aa, Homo sapiens
QDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 3 - DNA, 3072 bp, Mus musculus
gagaaccagaccaaggccctgtcatcagctcctggagactcagttctggtggcatggagaggacccttgtctgtctggtagtcatcttcttggggacagtggcccataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaagatgattcatcagcatctctcctagaacacataggacccgaagattcctgaggatccgagaagattcccgaggactgaggagacgccggacactatagacgctcacgaatgcaggagtacatcttgcctcttgggattgcaagtggagaagtacgatacgttatgataagaacaactcagaaaagctataggttaagatcctttcgcccattaactaagcagacattgtggttccctgcacagactccatgctgtcaacatggaaaatctcaactcaacaagagcccagcttcccgtgtcagggatttctggtgcttctcaagctgtggcttcatcttattgcccaactgtgacattctttgattggaaggggaaaactaaagcttttagcaaaaatacagctagggaatttgtcgatctgcgagagtaagacctcttatgatcctaacggaatgatgtaagctggaaataataagcataagatgaaattgaaaattgaagtctttattctttaagaaaaactttgtacttgaaagcatgtctgaagagtttactcattaccacaaacatctagcatattgataactaacatctttatactctacaagagaggctttccagataggtacagtttttcttctctattaggtctatcaaaatttaacctattatgagggtcacccctggctttcactgtttttctaaagaggcaagggtgtagtaagaagcaggcttaagttgccttcctcccaatgtcaagttcctttataagctaatagtttaatcttgtgaagatggcaatgaaagcctgtggaagtgcaaacctcactatcttctggagccaagtagaattttcaagtttgtagctctcacctcaagtggttatgggtgtcctgtgatgaatctgctagctccagcctcagtctcctctcccacatcctttcctttctttcctctttgaaacttctaagaaaaagcaatccaaacaagttcagcacttaagacacattgcatgcacacttttgataagttaaatccaaccatctatttaaaatcaaaatcaggagatgagccaagagaccagaggttctgttccagttttaaacagacttttactgaacatcccaatcttttaaccacagaggctaaattgagcaaatagttttgccatttgatataatttccaacagtatgtttcaatgtcaagttaaaaagtctacaaagctattttccctggagtggtatcatcgctttgagaatttcttatggttaaaatggatctgagatccaagcatggcctgggggatggttttgatctaaggaaaaaggtgtctgtacctcacagtgcctttaaaacaagcagagatcccgtgtaccgccctaagatagcacagactagtgttaactgattcccagaaaagtgtcacaatcagaaccaacgcattctcttaaactttaaaaatatgtattgcaaagaacttgtgtaactgtaaatgtgtgactgttgatgacattatacacacatagcccacgtaagtgtccaatggtgctagcattggttgctgagtttgctgctcgaaagctgaagcagagatgcagtccttcacaaagcaatgatggacagagaggggagtctccatgttttattcttttgttgtttctggctgtgtaactgttgacttcttgacattgtgatttttatatttaagacaatgtatttattttggtgtgtttattgttctagccttttaaatcactgacaatttctaatcaagaagtacaaataattcaatgcagcacaggctaagagcttgtatcgtttggaaaagccagtgaaggcttctccactagccatgggaaagctacgctttagagtaaactagacaaaattgcacagcagtcttgaacctctctgtgctcaagactcagccagtcctttgacattattgttcactgtgggtgggaacacattggacctgacacactgttgtgtgtccatgaaggttgccactggtgtaagctttttttggttttcattctcttatctgtagaacaagaatgtggggctttcctaagtctattctgtattttattctgaacttcgtatgtctgagttttaatgttttgagtactcttacaggaacacctgaccacacttttgagttaaattttatcccaagtgtgatatttagttgttcaaaaagggaagggatatacatacatacatacatacatacatacatatatatatatatatatacatatatatatatatatatatgtatatatatatatatatagagagagagagagagagagagagagaaagagagagaggttgttgtaggtcataggagttcagaggaaatcagttatggccgttaatactgtagctgaaagtgttttctttgtgaataaattcatagcattattgatctatgttattgctctgttttatttacagtcacacctgagaatttagttttaatatgaatgatgtactttataacttaatgattatttattatgtatttggttttgaatgtttgtgttcatggcttcttatttaagacctgatcatattaaatgctacccagtccgga

SEQ ID NO: 4 - protein, 122 aa, Mus musculus
PDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 5 - DNA, 2628 bp, Homo sapiens
gtcgacgcggcggtaccagctgtctgcccacttctcctgtggtgtgcctcacggtcacttgcttgtctgaccgcaagtctgcccatccctggggcagccaactggcctcagcccgtgccccaggcgtgccctgtctctgtctggctgccccagccctactgtcttcctctgtgtaggctctgcccagatgcccggctggtcctcagcctcaggactatctcagcagtgactcccctgattctggacttgcacctgactgaactcctgcccacctcaaaccttcacctcccaccaccaccactccgagtcccgctgtgactcccacgcccaggagaccacccaagtgccccagcctaaagaatggctttctgagaaagaccctgaaggagtaggtctgggacacagcatgccccggggcccactggctgccttactcctgctgattctccatggagcttggagctgcctggacctcacttgctacactgactacctctggaccatcacctgtgtcctggagacacggagccccaaccccagcatactcagtctcacctggcaagatgaatatgaggaacttcaggaccaagagaccttctgcagcctacacaggtctggccacaacaccacacatatatggtacacgtgccatatgcgcttgtctcaattcctgtccgatgaagttttcattgtcaatgtgacggaccagtctggcaacaactcccaagagtgtggcagctttgtcctggctgagagcatcaaaccagctccccccttgaacgtgactgtggccttctcaggacgctatgatatctcctgggactcagcttatgacgaaccctccaactacgtgctgaggggcaagctacaatatgagctgcagtatcggaacctcagagacccctatgctgtgaggccggtgaccaagctgatctcagtggactcaagaaacgtctctcttctccctgaagagttccacaaagattctagctaccagctgcaggtgcgggcagcgcctcagccaggcacttcattcagggggacctggagtgagtggagtgaccccgtcatctttcagacccaggctggggagcccgaggcaggctgggaccctcacatgctgctgctcctggctgtcttgatcattgtcctggttttcatgggtctgaagatccacctgccttggaggctatggaaaaagatatgggcaccagtgcccacccctgagagtttcttccagcccctgtacagggagcacagcgggaacttcaagaaatgggttaatacccctttcacggcctccagcatagagttggtgccacagagttccacaacaacatcagccttacatctgtcattgtatccagccaaggagaagaagttcccggggctgccgggtctggaagagcaactggagtgtgatggaatgtctgagcctggtcactggtgcataatccccttggcagctggccaagcggtctcagcctacagtgaggagagagaccggccatatggtctggtgtccattgacacagtgactgtgggagatgcagagggcctgtgtgtctggccctgtagctgtgaggatgatggctatccagccatgaacctggatgctggccgagagtctggccctaattcagaggatctgctcttggtcacagaccctgcttttctgtcttgcggctgtgtctcaggtagtggtctcaggcttggaggctccccaggcagcctactggacaggttgaggctgtcatttgcaaaggaaggggactggacagcagacccaacctggagaactgggtccccaggagggggctctgagagtgaagcaggttccccccctggtctggacatggacacatttgacagtggctttgcaggttcagactgtggcagccccgtggagactgatgaaggaccccctcgaagctatctccgccagtgggtggtcaggacccctccacctgtggacagtggagcccagagcagctagcatataataaccagctatagtgagaagaggcctctgagcctggcatttacagtgtgaacatgtaggggtgtgtgtgtgtgtgtgtgtgtgtgtgtgtgtgtgtgtgtgtgtgtgtgtgtgtgtgtgtcttgggttgtgtgttagcacatccatgttgggatttggtctgttgctatgtattgtaatgctaaattctctacccaaagttctaggcctacgagtgaattctcatgtttacaaacttgctgtgtaaaccttgttccttaatttaataccattggttaaataaaattggctgcaaccaattactggagggattagaggtagggggcttttgagttacctgtttggagatggagaaggagagaggagagaccaagaggagaaggaggaaggagaggagaggagaggagaggagaggagaggagaggagaggagaggagaggagaggagaggctgccgtgaggggagagggaccatgagcctgtggccaggagaaacagcaagtatctggggtacactggtgaggaggtggccaggccagcagttagaagagtagattaggggtgacctccagtatttgtcaaagccaattaaaataacaaaaaaaaaaaaaaagcggccgctctaga

SEQ ID NO: 6 - protein, 538 aa, Homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 7 - DNA, 2665 bp, Mus musculus
gtcgactggaggcccagctgcccgtcatcagagtgacaggtcttatgacagcctgattggtgactcgggctgggtgtggattctcaccccaggcctctgcctgctttctcagaccctcatctgtcacccccacgctgaacccagctgccacccccagaagcccatcagactgcccccagcacacggaatggatttctgagaaagaagccgaaacagaaggcccgtgggagtcagcatgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatgaggctgactggatgtccagagctggccaggccactgggccctgagccagagacaaggtcacctgggctgtgatgtgaagacacctgcagcctttggtctcctggatgggcctttgagcctgatgtttacagtgtctgtgtgtgtgtgtgcatatgtgtgtgtgtgcatatgcatgtgtgtgtgtgtgtgtgtcttaggtgcgcagtggcatgtccacgtgtgtgtgtgattgcacgtgcctgtgggcctgggataatgcccatggtactccatgcattcacctgccctgtgcatgtctggactcacggagctcacccatgtgcacaagtgtgcacagtaaacgtgtttgtggtcaacagatgacaacagccgtcctccctcctagggtcttgtgttgcaagttggtccacagcatctccggggctttgtgggatcagggcattgcctgtgactgaggcggagcccagccctccagcgtctgcctccaggagctgcaagaagtccatattgttccttatcacctgccaacaggaagcgaaaggggatggagtgagcccatggtgacctcgggaatggcaattttttgggcggcccctggacgaaggtctgaatcccgactctgataccttctggctgtgctacctgagccaagtcgcctcccctctctgggctagagtttccttatccagacagtggggaaggcatgacacacctgggggaaattggcgatgtcacccgtgtacggtacgcagcccagagcagaccctcaataaacgtcagcttccttcaaaaaaaaaaaaaaaaatctaga

SEQ ID NO: 8 - protein, 529 aa, Mus musculus
MPRGPVAALLLLILHGAWSCLDLTCYTDYLWTITCVLETRSPNPSILSLTWQDEYEELQDQETFCSLHRSGHNTTHIWYTCHMRLSQFLSDEVFIVNVTDQSGNNSQECGSFVLAESIKPAPPLNVTVAFSGRYDISWDSAYDEPSNYVLRGKLQYELQYRNLRDPYAVRPVTKLISVDSRNVSLLPEEFHKDSSYQLQVRAAPQPGTSFRGTWSEWSDPVIFQTQAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPRSYLRQWVVRTPPPVDSGAQSS

SEQ ID NO: 9 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 10 - protein, 123 aa, Peromyscus maniculatus
VVIFLGTVAHKTSPQRPDRLLIRLRHLVDNVEQLKIYVNDLDPELLPAPQDVKEHCAHSAFACFQKAKLKPANTGSNKTIISDLVTQLRRRLPATKAEKKQQSLVKCPSCDSYEKKTPKEFLE

SEQ ID NO: 11 - protein, 146 aa, Mus musculus [sequence omitted]

SEQ ID NO: 12 - protein, 152 aa, Bos taurus
MRWPGNMERIVICLMVIFSGTVAHKSSSQGQDRLFIRLRQLIDIVDQLKNYVNDLDPEFLPAPEDVKRHCERSAFSCFQKVQLKSANNGDNEKIINILTKQLKRKLPATNTGRRQKHEVTCPSCDSYEKKPPKEYLERLKSLIQKMIHQHLS

(19) United States
(12) Patent Application Publication
(10) Pub. No.: US 2006/0024268 A1
(43) Pub. Date: Feb. 2, 2006

(54) Modulation of immunoglobulin production and atopic disorders

(75) Inventors: Marion T. Kasaian, Charlestown, MA (US); Debra D. Donaldson, Medford, MA (US); Mary Collins, Natick, MA (US); Nancy L. Wood, Somerville, MA (US)

Correspondence Address: FITZPATRICK CELLA HARPER & SCINTO, 30 ROCKEFELLER PLAZA, NEW YORK, NY, 10112, US

(73) Assignee: WYETH

(21) Appl. No.: 11/132,947

(22) Filed: May 19, 2005

Related U.S. Application Data
(60) Provisional application No. 60/572,407, filed on May 19, 2004.

(51) Int. Cl. A61K 38/20

(52) U.S. Cl. 424/85.2

(57) ABSTRACT

An IL-21 polypeptide or other IL-21 pathway agonist can be used to treat atopic disorders, e.g., asthma.

### CROSS-REFERENCE TO RELATED APPLICATIONS

This application claims priority to U.S. Application Ser. No. 60/572,407, filed on May 19, 2004, the contents of which are hereby incorporated by reference.

### BACKGROUND

IgE generated in response to allergen challenge triggers potent agonist mechanisms associated with atopic disease. When bound to high affinity receptors on mast cells and basophils, IgE can be cross-linked by allergen, leading to degranulation and the release of histamine, leukotrienes, and other inflammatory mediators. These agents directly mediate the symptoms of wheezing, bronchoconstriction, and rhinitis associated with early and late phase allergic reactions, while cytokines and chemokines released by mast cells and basophils contribute to local inflammatory reactions. The central role of IgE in these responses is supported not only by the detection of allergen-specific IgE in atopic subjects compared to healthy controls, but also by the demonstration that neutralization of IgE is an effective therapeutic strategy for the treatment of atopic disease. See, e.g., Kawakami and Galli (2002) Nat Rev Immunol 2(10); 773-86; Prussin and Metcalfe (2003) J Allergy Clin Immunol 111(2 Suppl); S486-94; Holgate (2000) Clin Exp Allergy 30 Suppl 1; 28-32; Busse and Neaville, (2001) Curr Opin Allergy Clin Immunol 1(1); 105-8.

### SUMMARY

We have discovered, inter alia, that IL-21 polypeptide can generate a protective environment against atopic reactions. Accordingly, IL-21 pathway agonists, such as IL-21 polypeptide and other agents that similarly regulate the IL-21 pathway, can be used to regulate the balance between IgE and IgG4 produced in response to allergen exposure. For example, IL-21 pathway agonists can be used to reduce levels or production of IgE in a subject, ameliorate at least one symptom of an atopic disorder, and/or inhibit production of IgE in a subject.

In one aspect, the invention features a method of ameliorating one or more symptoms associated with an atopic disorder in a subject. The method includes: administering, to the subject, an IL-21 pathway agonist, in an amount effective for ameliorating one or more symptoms of the atopic disorder. Exemplary atopic disorders include: atopic dermatitis, asthma, extrinsic bronchial asthma, urticaria, eczema, allergic rhinitis, and allergic enterogastritis.

The term “IL-21 pathway” refers to the biological components that mediate IL-21 signaling. The pathway includes, e.g., IL-21 polypeptide itself, IL-21 receptor, and cytoplasmic components that are modulated by receptor activation, including STAT3 and STAT5, kinases, and/or transcription factors. The term “IL-21 pathway agonist” refers to an agent that increases activity of the IL-21 pathway, e.g., an agent that potentiates, induces or otherwise enhances one or more biological activities of an IL-21 receptor polypeptide, e.g., a biological activity as described herein. For example, an agonist interacts with, e.g., binds to, an IL-21 receptor polypeptide. In one embodiment, an agonist may interact with IL-21 receptor and another receptor chain, e.g., the γ cytokine receptor chain. For example, the agonist crosslinks IL-21 receptor and γ cytokine receptor chain.

In one embodiment, the IL-21 pathway agonist is an IL-21 polypeptide, an active fragment or a variant thereof. For example, the IL-21 polypeptide is administered in a dose of about 0.1 μg to about 100 μg, about 100 μg to about 5 mg or about 5 mg to about 100 mg per kg body weight. The IL-21 polypeptide can be, e.g., human or substantially human. The IL-21 polypeptide can include the amino acid sequence of SEQ ID NO:2 or an amino acid sequence that is at least 85, 90, 92, 94, 95, 96, 97, 98, or 99% identical to SEQ ID NO:2.

In another embodiment, the IL-21 pathway agonist is an agent that interacts with the IL-21 receptor. An agent that interacts with the IL-21 receptor can activate the receptor or otherwise agonize pathway signaling. For example, the IL-21 pathway agonist is a protein that interacts with the IL-21 receptor. The protein can comprise an agonistic anti-IL-21 receptor antibody (e.g., a full length antibody or an antigen-binding fragment) that interacts with and activates the IL-21 receptor.

In one embodiment, the IL-21 pathway agonist is an agent that modulates a cytoplasmic IL-21 pathway component. An agent that modulates a cytoplasmic IL-21 pathway component can, for example, activate a positively acting cytoplasmic pathway component or inhibit a negatively acting cytoplasmic component. Exemplary positively acting cytoplasmic components include the STAT kinases. The agent may also be a mimic of a positively acting component, e.g., a constitutively activated form of a STAT kinase.

In one embodiment, the IL-21 pathway agonist is a nucleic acid that encodes an IL-21 polypeptide, a protein that interacts with (e.g., binds and/or activates) the IL-21 receptor, and a protein that modulates a cytoplasmic IL-21 pathway component. The agent may encode a positively acting component, e.g., a nucleic acid encoding a STAT kinase or a constitutively activated form of a STAT kinase.

The subject can be mammalian, and typically is human (e.g., a female or a male, and an adult or a juvenile human subject). IgE levels in the subject can be decreased by at least 10, 20, 30, 40, 50, 70, 80, 85, 90, or 95% relative to a reference parameter, either locally or systemically. For example, the reference parameter can be a parameter for the subject prior to treatment or can be a normal or control subject or a statistical value characteristic of a population of subjects (e.g., a cohort of normal subjects, e.g., of similar age and gender).

The IL-21 pathway agonist can be administered parenterally or locally. For example, the agonist can be delivery topically to a site of an atopic dermatitis. It can be delivered to respiratory mucosa, e.g., by inhalation, e.g., of an atomized composition. It can be delivered parenterally, e.g., by injection, e.g., subcutaneous, intramuscular, or intravenous. It can be delivered, e.g., by an implant or other medical device. Other exemplary modes are described herein.

The method can further include evaluating one or more symptoms of the atopic disorder in the subject, e.g., before, during, or after the administering. Examples of such symptoms are described herein. The method can further include evaluating an IL-21 associated parameter in the subject, e.g., a parameter associated with level of IL-21 polypeptide, IL-21 receptor, or IL-21 pathway activity. The term “parameter” refers to information, including qualitative and quantitative descriptors, e.g., values, levels, measurements, and so forth. An “IL-21 associated parameter” refers to a parameter that describes an IL-21 pathway component, e.g., the presence, absence, level, expression, stability, subcellular localization, or activity of such a component, e.g., an IL-21 polypeptide, an IL-21 receptor, or other cytoplasmic component. The parameter may also describe an mRNA that encodes an IL-21 pathway component.

The method can further include evaluating an endogenous immunoglobulin (e.g., IgG or IgE) in the subject, e.g., evaluating levels of the endogenous immunoglobulin.

The method can include other features described herein.

In another aspect, the invention a method of treating or preventing an atopic disorder in a subject, the method including: administering, to the subject, an IL-21 pathway agonist, in an amount effective for treating or preventing the atopic disorder. Exemplary atopic disorders include: atopic dermatitis, asthma, extrinsic bronchial asthma, urticaria, eczema, allergic rhinitis, and allergic enterogastritis.

In one embodiment, the IL-21 pathway agonist is an IL-21 polypeptide. For example, the IL-21 polypeptide is administered in a dose of about 0.1 μg to about 100 μg, about 100 μg to about 5 mg or about 5 mg to about 100 mg per kg body weight. The IL-21 polypeptide can be, e.g., human or substantially human. The IL-21 polypeptide can include the amino acid sequence of SEQ ID NO:2 or an amino acid sequence that is at least 85, 90, 92, 94, 95, 96, 97, 98, or 99% identical to SEQ ID NO:2.

In one embodiment, the IL-21 pathway agonist is an agent that interacts with the IL-21 receptor, an agent that modulates a cytoplasmic IL-21 pathway component or a nucleic acid that encodes an IL-21 polypeptide, a protein that interacts with (e.g., activates) the IL-21 receptor, and a protein that modulates a cytoplasmic IL-21 pathway component.

The subject can be mammalian, and typically is human (e.g., a female or a male, and an adult or a juvenile human subject). IgE levels in the subject can be decreased by at least 10, 20, 30, 40, 50, 70, 80, 85, 90, or 95% relative to a reference parameter, either locally or systemically. For example, the reference parameter can be a parameter for the subject prior to treatment or can be a normal or control subject or a statistical value characteristic of a population of subjects (e.g., a cohort of normal subjects, e.g., of similar age and gender).

The IL-21 pathway agonist can be administered parenterally or locally. For example, the agonist can be delivery topically to a site of an atopic dermatitis. It can be delivered to respiratory mucosa, e.g., by inhalation, e.g., of an atomized composition. It can be delivered parenterally, e.g., by injection, e.g., subcutaneous, intramuscular, or intravenous. It can be delivered, e.g., by an implant or other medical device. Other exemplary modes are described herein.

The method can include other features described herein.

In another aspect, the invention features a method of modulating IgG production in a cell (e.g., a B cell, e.g., a mammalian, e.g., human, murine, or other rodent cell). The method includes: contacting an IL-21 pathway modulator, to the cell in an amount sufficient to modulate IgG production (e.g., expression or secretion from a cell). The cell can be in vitro or in vivo during the contacting step. For example, in vivo contacting can be performed in a mammalian subject, e.g., a human subject.

In one embodiment, IgG production is increased and the IL-21 pathway modulator is an IL-21 pathway agonist, e.g., an IL-21 polypeptide, an agent that interacts with the IL-21 receptor, or an agent that modulates a cytoplasmic IL-21 pathway component. IgG levels can be increased, e.g., by at least 10, 20, 30, 40, 50, 70, 80, 100, 120, or 150% relative to a reference parameter. For example, the reference parameter can be a parameter for the subject prior to treatment or can be a normal or control subject or a statistical value characteristic of a population of subjects (e.g., a cohort of normal subjects, e.g., of similar age and gender).

In another embodiment, IgG production is decreased and the IL-21 pathway modulator is an IL-21 pathway antagonist. IgG levels can be decreased, e.g., by at least 10, 20, 30, 40, 50, 70, 80, 85, 90, or 95% relative to a reference parameter (e.g., a parameter for the subject prior to treatment or can be a normal or control subject or a statistical value characteristic of a population of subjects (e.g., a cohort of normal subjects, e.g., of similar age and gender)).

In a first example, the antagonist is an agent that binds to IL-21 or an IL-21 receptor, such as an antibody or antigen-binding fragment thereof that binds IL-21 or an agent that includes a soluble form of the IL-21 receptor, e.g., an extracellular domain thereof (e.g., an extracellular domain alone or as a fusion such as an Fc fusion). In a second example, the IL-21 pathway antagonist is an agent that binds to a component of the IL-21 receptor, e.g., and the agent prevents activation of the IL-21 receptor. An antibody that binds to IL-21 receptor and prevents binding of IL-21 to the receptor is one agent that has these properties. In a third example, the IL-21 pathway antagonist is a nucleic acid (e.g., an anti-sense RNA, an siRNA, or a ribozyme) that reduces expression of IL-21, IL-21 receptor, or an IL-21 pathway component.

The method can include other features described herein.

In another aspect, the invention features a method of modulating IgE production in a cell. The method includes: contacting an IL-21 pathway modulator, to the cell in an amount sufficient to modulate IgE production. The term “IL-21 pathway modulator” refers to an agent that alters activity of the IL-21 pathway and encompasses IL-21 pathway agonists and antagonists.

In one embodiment, IgE production is decreased and the IL-21 pathway modulator is an IL-21 pathway agonist, e.g., an agonist described herein, e.g., an IL-21 polypeptide. For example, IgE levels are decreased by at least 10, 20, 30, 40, 50, 70, 80, 85, 90, or 95% relative to a reference parameter (e.g., a parameter for the subject prior to treatment or can be a normal or control subject or a statistical value characteristic of a population of subjects (e.g., a cohort of normal subjects, e.g., of similar age and gender)).

In another embodiment, IgE production is increased and the IL-21 pathway modulator is an IL-21 pathway antagonist, e.g., an antagonist described herein. For example, the levels are increased by at least 10, 20, 30, 40, 50, 70, 80, 100, 120, or 150% relative to a reference parameter (e.g., a parameter for the subject prior to treatment or can be a normal or control subject or a statistical value characteristic of a population of subjects (e.g., a cohort of normal subjects, e.g., of similar age and gender)). The method can include other features described herein.

In another aspect, the invention features method of modulating relative levels of IgE and IgG, the method including: contacting an IL-21 pathway modulator, to the cell in an amount sufficient to modulate relative levels of IgE and IgG.

In one embodiment, the IgE/IgG ratio is decreased and the IL-21 pathway modulator is an IL-21 pathway agonist, e.g., an agonist described herein, e.g., an IL-21 polypeptide. For example, the ratio is decreased by at least 10, 20, 30, 40, 50, 70, 80, 85, 90, or 95% relative to a reference ratio (e.g., a ratio for the subject prior to treatment or can be a normal or control subject or a statistical value characteristic of a population of subjects (e.g., a cohort of normal subjects, e.g., of similar age and gender)).

In another embodiment, the IgE/IgG ratio is increased and the IL-21 pathway modulator is an IL-21 pathway antagonist, e.g., an antagonist described herein. For example, the ratio is increased by at least 10, 20, 30, 40, 50, 70, 80, 100, 120, or 150% relative to a reference ratio (e.g., a ratio for the subject prior to treatment or can be a normal or control subject or a statistical value characteristic of a population of subjects (e.g., a cohort of normal subjects, e.g., of similar age and gender)).

It is possible to modulate the relative levels of IgE and IgG by inhibiting a switch recombination required for the Iε transcript. These relative levels may also be modulated in the presence of T cells.

In still another aspect, the invention features a pharmaceutical composition that includes an IL-21 pathway agonist and a second agent for treating an atopic disorder. In one embodiment, the IL-21 pathway agonist is an IL-21 polypeptide. For example, the IL-21 polypeptide is administered in a dose of about 0.1 μg to about 100 μg, about 100 μg to about 5 mg or about 5 mg to about 100 mg per kg body weight. The IL-21 polypeptide can be, e.g., human or substantially human. The IL-21 polypeptide can include the amino acid sequence of SEQ ID NO:2 or an amino acid sequence that is at least 85, 90, 92, 94, 95, 96, 97, 98, or 99% identical to SEQ ID NO:2.

In one embodiment, the IL-21 pathway agonist is an agent that interacts with the IL-21 receptor, an agent that modulates a cytoplasmic IL-21 pathway component or a nucleic acid that encodes an IL-21 polypeptide, a protein that interacts with (e.g., activates) the IL-21 receptor, and a protein that modulates a cytoplasmic IL-21 pathway component.

In another aspect, the invention features a container that includes one or more doses of a pharmaceutical composition of an IL-21 pathway agonist and a label, the label including instruction for administering a dose of the composition for treating or preventing an atopic disease or disorder. In one embodiment, the composition includes a second agent for treating an atopic disorder.

The invention also includes a method for manufacturing a pharmaceutical. The method includes providing an IL-21 pathway agonist and packaging the agonist in a container. The method can also include associating (e.g., affixing) a label to the container, e.g., a label that includes instructions for treating or preventing an atopic disease or disorder. In one embodiment, the IL-21 pathway agonist is an IL-21 polypeptide. The method can include recombinantly expressing the IL-21 polypeptide and at least partially purifying the polypeptide.

In another aspect, the invention features a method of evaluating a subject having or suspected of having an atopic disorder, e.g., atopic dermatitis, asthma, extrinsic bronchial asthma, urticaria, eczema, allergic rhinitis, and allergic enterogastritis. The method includes: evaluating an IL-21 associated parameter for a subject having an atopic disorder, comparing results of the evaluating to a reference parameter, and providing a recommendation of a therapy for the disorder as a function of the comparison. A “reference parameter” refers to corresponding information from a reference subject or cell, e.g., a control, normal, or wild-type subject or cell. A reference parameter may also be the average or median of a control group or normal group of individuals. For example, the IL-21 associated parameter includes a quantitative or qualitative value for IL-21 polypeptide abundance or IL-21 mRNA abundance. In another example, the IL-21 associated parameter includes a quantitative or qualitative value for IL-21 receptor protein or mRNA, or for an IL-21 pathway activity. The recommended therapy can include administration of an IL-21 pathway agonist, e.g., an IL-21 polypeptide. The method can include other features described herein.

In another aspect, the invention features a method of evaluating a subject for risk of an atopic disorder. The method includes: evaluating an IL-21 associated parameter for a subject, comparing results of the evaluating to a reference parameter, and providing a risk assessment for an atopic disorder as a function of the comparison. For example, the risk assessment can be a function of the deviation between the evaluated parameter and the reference parameter. In one embodiment, the risk assessment is expressed as the number of standard deviations from the norm. The method can include other features described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. U.S. application Ser. No. 10/806,611, filed on Mar. 22, 2004, and U.S. 2003-0108549 are hereby incorporated by reference in their entireties. In the case of conflict, the present specification, including definitions, controls. In addition, the materials, methods, and examples are illustrative only and not intended to be limiting.

### DESCRIPTION OF THE DRAWINGS

FIG. 1. IL-21 potentiates IgE and IgG4 release from purified B cells. B cells were isolated from human PBMC by magnetic bead separation. Cells were treated with anti-CD40 plus the indicated cytokines as described in Materials and Methods. On day 6, cells and supernatants were harvested. (A, B) IgE levels in supernatants of individual microwells. (C) PCR for expression of GAPDH, Iε sterile transcript, and Iγ4 sterile transcript. (D) IgG4 levels in pooled wells treated with the indicated cytokine. No IgG4 was detectable in cells treated with anti-CD40 alone.

FIG. 2. IL-21 synergizes with IL-4 or IL-13 to drive B cell proliferation. B cells were isolated from human PBMC by magnetic bead separation. Cells were treated for 48 hours with anti-CD40 plus the indicated cytokines. 3H-thymidine was added for the final 24 hours, and incorporation determined by liquid scintillation counting.

FIG. 3. IL-21 potentiates IgE and IgG4 release from PBMC stimulated with anti-CD40. Unfractionated human PBMC were treated with anti-CD40 plus the indicated cytokines, as described in Materials and Methods. (A) IgE levels in supernatants of individual microwells, assayed on day 21 of culture. There was no detectable IgE in wells treated with IL-21 alone. (B) IgE levels in pooled wells treated with the indicated cytokine, assayed on day 21 of culture. (C) PCR for Iε and Iγ4 sterile transcripts was performed using cells isolated on day 3 of culture. PCR for Cε mature transcript was performed using cells isolated on day 10. (D) IgG4 levels in pooled wells treated with the indicated cytokine, assayed on day 21 of culture.

FIG. 4. IL-21 inhibits IgE production but not IgG4 release in PBMC stimulated with PHA. Unfractionated human PBMC were treated with PHA and cytokines. (A) IgE levels in supernatants of individual microwells, assayed on day 21 of culture. (B) IgE levels in pooled wells treated with the indicated cytokine, assayed on day 21 of culture. (C) PCR for Iε and Iγ4 sterile transcripts, using cells isolated on day 3 of culture. (D) IgG4 levels in pooled wells treated with the indicated cytokine, assayed on day 21 of culture.

FIG. 5 (A,B) shows changes to CD40L expression as described infra.

FIG. 6. Cytokine levels in PBMC cultures. (A) Unfractionated PBMC were treated with PHA and cytokines as described in Materials and Methods. IL-10 levels were measured in pooled supernatants collected on day 7 of culture. (B) Unfractionated human PBMC were treated for 48 hours with anti-CD40 plus the indicated cytokines. On day 2 and every 4 days thereafter, media was changed and fresh cytokines added. IL-10 levels were measured in pooled supernatants collected on day 7. (C) PHA-stimulated PBMC were treated with the indicated cytokines. IL-12 levels were measured in pooled supernatants collected on day 6 of culture. (D) PHA-stimulated PBMC were treated with the indicated cytokines. IL-12Rb transcripts were quantitated by real-time PCR in cells collected on day 6 of culture. Data are expressed as Relative TAQMAN™ Units (RTU).

FIG. 7 shows changes in apoptotic CD19⁺ cell number as described infra.

FIG. 8. IL-13 does not rescue IgE production from PHA-stimulated PBMC treated with IL-4 and IL-21. Unfractionated human PBMC were treated with PHA and cytokines. IgE levels were determined in pooled wells treated with the indicated cytokine, assayed on day 14 of culture. (A) Effects of IL-21 and IL-13 on IL-4 driven IgE production. (B) Effects of IL-21 and IL-4 on IL-13 driven IgE production.

FIG. 9 shows changes to IgE levels under various conditions.

FIG. 10. IL-21 does not reduce IgE production in irradiated PBMC. Unfractionated PBMC were: (A) irradiated; or (B) not irradiated. The cells were stimulated with PHA for 2 days at 37° C., then treated with IL-4+/−IL-21, as described in Materials and Methods. IgE levels were measured in pooled supernatants collected on day 13 of culture. Data are expressed as percentage of IgE levels found in the IL-4 stimulated cultures.

### DETAILED DESCRIPTION

IL-21 is a cytokine that regulates immune cell behavior. We have discovered that IL-21 can be used to modulate IgE production. Reactivity caused by IgE contributes to a number of disorders, including atopic disorders. Use of a IL-21 polypeptide or a similarly acting IL-21 pathway agonist can be used, for example, to decrease IgE levels, locally or systemically in a subject, thereby ameliorating the atopic disorder.

IL-21 Pathway Agonists

In one aspect of the invention, IL-21 pathway agonists are used to modulate the immune system, for example, to treat, prevent, or ameliorate an atopic disorder. Exemplary IL-21 pathway agonists include a IL-21 polypeptide, IL-21 receptor, agents that activate or agonize IL-21 receptor, and agents that modulate other IL-21 pathway components to activate IL-21 pathway signaling. Exemplary agonists bind to IL-21 polypeptide or IL-21 receptor with high affinity, e.g., with an affinity constant of less than about 10⁷M⁻¹, about 10⁸ M⁻¹, or, about 10⁹M⁻¹ to 10¹⁰ M⁻¹ or stronger.

Exemplary IL-21 pathway components include IL-21 polypeptide, IL-21 receptor, receptor β chain, the common γ cytokine chain), and intracellular signaling components, such as Jak1, Jak3, STAT1, STAT3, and STAT5.

IL-21

In its mature form, the human IL-21 cytokine is about 131-amino acids in length and has sequence homology to IL-2, IL-4 and IL-15 (Parrish-Novak et al. (2000) Nature 408:57-63). Despite low sequence homology among interleukin cytokines, these cytokines and IL-21 share a common fold that includes a characteristic “four-helix-bundle” structure.

Amino acid sequences of IL-21 polypeptides are publicly known. For example, the nucleotide sequence and amino acid sequence of a human IL-21 is available at GENBANK® Acc. No. X_{—}011082. An exemplary disclosed human IL-21 nucleotide sequence is presented below:

| (SEQ ID NO:1) | |
|---|---|
| 1 | gctgaagtga aaacgagacc aaggtctagc tctactgttg gtacttatga gatccagtcc |
| 61 | tggcaacatg gagaggattg tcatctgtct gatggtcatc ttcttgggga cactggtcca |
| 121 | caaatcaagc tcccaaggtc aagatcgcca catgattaga atgcgtcaac ttatagatat |
| 181 | tgttgatcag ctgaaaaatt atgtgaatga cttggtccct gaatttctgc cagctccaga |
| 241 | agatgtagag acaaactgtg agtggtcagc tttttcctgc tttcagaagg cccaactaaa |
| 301 | gtcagcaaat acaggaaaca atgaaaggat aatcaatgta tcaattaaaa agctgaagag |
| 361 | gaaaccacct tccacaaatg cagggagaag acagaaacac agactaacat gcccttcatg |
| 421 | tgattcttat gagaaaaaac cacccaaaga attcctagaa agattcaaat cacttctcca |
| 481 | aaagatgatt catcagcatc tgtcctctag aacacacgga agtgaagatt cctgaggatc |
| 541 | taacttgcag ttggacacta tgttacatac tctaatatag tagtgaaagt catttctttg |
| 601 | tattccaagt ggaggag |

Additional nucleotide sequence information is available, e.g., from AF254069 [gi:11093535] which provides a 642 bp mRNA sequence encoding an exemplary IL-21 polypeptide. In some embodiments, it is sufficient to use the region of nucleotide sequence that encodes mature IL-21, e.g., without a region encoding a signal sequence. The amino acid sequence of an exemplary mature human IL-21 polypeptide, based on Parrish-Novak et al. (2000) Nature 408:57-63, is presented below:

(SEQ ID NO:2)

QDRHMIRMRQLIDIVDQLKNYVNDLVPEFLPAPEDVETNCEWSAFSCFQK
AQLKSANTGNNERIINVSIKKLKRKPPSTNAGRRQKHRLTCPSCDSYEKK
PPKEFLERFKSLLQKMIHQHLSSRTHGSEDS

The full length sequence of an exemplary human IL-21 polypeptide is:

(SEQ ID NO: 9)

MRSSPGNNERIVICLMVIFLGTLVHKSSSQGQDRHMIRMRQLIDIVDQLK
NYVNDLVPEFLPAPEDVETNCEWSAFSCFQKAQLKSANTGNNERIINVSI
KKLKRKPPSTNAGRRQKHRLTCPSCDSYEKKPPKEFLERFKSLLQKMIHQ
HLSSRTHGSEDS

Additional entries providing amino acid sequences for human IL-21 polypeptides are as follows: gi|11141875|ref|NP_{—}068575.1|interleukin 21 [Homo sapiens]; gi|11093536|gb|AAG29348.1|interleukin 21 [Homo sapiens]; gi|42542586|gb|AAH66259.1|Interleukin 21 [Homo sapiens]; gi|42542588|gb|AAH66260.1|Interleukin 21 [Homo sapiens]; gi|42542657|gb|AAH66261.1|Interleukin 21 [Homo sapiens]; gi|42542659|gb|AAH66258.1|Interleukin 21 [Homo sapiens]; and gi|42542807|gb|AAH66262.1|Interleukin 21 [Homo sapiens]. The human IL-21 polypeptide can be a variant of a polypeptide described herein, provided that it retains functionality.

Exemplary IL-21 polypeptides from other species include the following:

interleukin-21 from Peromyscus maniculatus:

(SEQ ID NO:10)

VVIFLGTVAHKTSPQRPDRLLIRLRHLVDNVEQLKIYVNDLDPELLPAPQ
DVKEHCAHSAFACFQKAKLKPANTGSNKTIISDLVTQLRRRLPATKAEKK
QQSLVKCPSCDSYEKKTPKEFLE

interleukin-21 from Mus musculus:

(SEQ ID NO:4, mature form)

PDRLLIRLRHLIDIVEQLKIYENDLDPELLSAPQDVKGHCEHAAFACFQK
AKLKPSNPGNNKTFIIDLVAQLRRRLPARRGGKKQKHIAKCPSCDSYEKR
TPKEFLERLKWLLQKMIHQHLS,

(SEQ ID NO:11, full length)

MERTLVCLVVIFLGTVAIHKSSPQGPDRLLIRLRHLIDIVEQLKIYENDL
DPELLSAPQDVKGHCEHAAFACFQKAKLKPSNPGNNKTFIIDLVAQLRRR
LPARRGGKKQKHIAKCPSCDSYEKRTPKEFLERLKWLLQKMIHQHLS

interleukin-21 from Bos taurus:

(SEQ ID NO:12)

MRWPGNMERIVICLMVIFSGTVAHKSSSQGQDRLFIRLRQLIDIVDQLKN
YVNDLDPEFLPAPEDVKRHCERSAFSCFQKVQLKSANNGDNEKIINILTK
QLKRKLPATNTGRRQKHEVTCPSCDSYEKKPPKEYLERLKSLIQKMIHQH
LS

The terms “interleukin-21”, “IL-21” and “IL-21 polypeptide” refer to a protein (e.g., a mammalian, e.g., murine or human protein) which is capable of interacting with, e.g., binding to, IL-21 receptor (e.g., a mammalian, e.g., murine or human protein) and having one of the following features: (i) an amino acid sequence of a naturally occurring mammalian IL-21 or a fragment thereof, e.g., an amino acid sequence shown as SEQ ID NO:2 (human, mature), SEQ ID NO:9 (human, full length), SEQ ID NO:10 (Peromyscus), SEQ ID NO:12 (Bos), SEQ ID NO:4 (murine, mature), or SEQ ID NO:11 (murine, full length) or a fragment thereof; (ii) an amino acid sequence substantially homologous to, e.g., at least 85%, 90%, 95%, 98%, 99% homologous to, an amino acid sequence shown as SEQ ID NO:2 (human, mature), SEQ ID NO:9 (human, full length), SEQ ID NO:10 (Peromyscus), SEQ ID NO:12 (Bos), SEQ ID NO:4 (murine, mature), or SEQ ID NO:11 (murine, full length) or a fragment thereof; (iii) an amino acid sequence which is encoded by a naturally occurring mammalian IL-21 nucleotide sequence or a fragment thereof (e.g., SEQ ID NO:1 (human) or SEQ ID NO:3 (murine), or a fragment thereof, e.g., a region encoding a mature form); (iv) an amino acid sequence encoded by a nucleotide sequence which is substantially homologous to, e.g., at least 85%, 90%, 95%, 98%, 99% homologous to, a nucleotide sequence shown as SEQ ID NO:1 (human) or SEQ ID NO:3 (murine), or a fragment thereof (e.g., a region encoding a mature form); (v) an amino acid sequence encoded by a nucleotide sequence degenerate to a naturally occurring IL-21 nucleotide sequence or a fragment thereof, e.g., SEQ ID NO:1 (human) or SEQ ID NO:3 (murine), or a fragment thereof (e.g., a region encoding a mature form); or (vi) an amino acid sequence, of at least 115 amino acids that is encoded by a nucleotide sequence that hybridizes to the complement of one of the foregoing nucleotide sequences under stringent conditions, e.g., highly stringent conditions (for example, the nucleotide sequence hybridizes in a region that encodes a mature IL-21 protein). IL-21 binding to IL-21 receptor can lead to STAT5 or STAT3 signaling (Ozaki et al. (2000) Proc. Natl. Acad. Sci. USA 97:11439-11444). IL-21 polypeptide can be processed from a nascent protein that includes a signal sequence to a mature protein, from which the signal sequence has been removed.

Sequences similar or homologous (e.g., at least about 85% sequence identity) to the sequences disclosed herein are also part of this application. In some embodiment, the sequence identity can be about 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. The comparison uses the GAP program from the GCG software package (www.gcg.com) and parameters that include a Blossum 62 scoring matrix with a gap penalty of 12, a gap extend penalty of 4, and a frameshift gap penalty of 5.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, example of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6×SSC at about 45° C., followed by one or more washes in 0.2×SSC, 0.1% SDS at 55° C. A further example of stringent hybridization conditions are hybridization in 6×SSC at about 45° C., followed by one or more washes in 0.2×SSC, 0.1% SDS at 60° C. Preferably, stringent hybridization conditions are hybridization in 6×SSC at about 45° C., followed by one or more washes in 0.2×SSC, 0.1% SDS at 65° C. Particularly preferred highly stringent conditions (and the conditions that should be used if the practitioner is uncertain about what conditions should be applied to determine if a molecule is within a hybridization limitation) are 0.5M sodium phosphate, 7% SDS at 65° C., followed by one or more washes at 0.2×SSC, 1% SDS at 65° C.

An IL-21 polypeptide may have additional conservative or non-essential amino acid substitutions, which do not have a substantial effect on their functions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In one embodiment, the IL-21 polypeptide is substantially human. An “substantially human” IL-21 polypeptide is an IL-21 polypeptide that includes a sufficient number of human amino acid positions such that the polypeptide does not elicit an immunogenic response in a normal human and so that the IL-21 polypeptide interacts with a human IL-21 receptor.

Forms of IL-21 polypeptides less than full length can be used in the methods and compositions, described herein, provided that such form retains the ability to bind to an IL-21 receptor. In one embodiment, the form is a functional IL-21 polypeptide, e.g., a form that can activate IL-21 pathway signaling.

IL-21 polypeptides of less than full length can be produced, for example, by expressing a corresponding fragment of the polynucleotide encoding the full-length IL-21 protein in a host cell, or by expressing a polynucleotide encoding a modified protein (e.g., if one or more internal amino acids are removed). One form of IL-21 polypeptide that is less than full length is mature IL-21, e.g., an IL-21 of SEQ ID NO:2. Another form is a polypeptide that is shorter than a full-length, mature IL-21, e.g., less than 131, 130, 129, 128, or 125 amino acids, e.g., between 115 and 130 amino acids in length. For example, an IL-21 polypeptide derived from SEQ ID NO:2 can be missing the final eight amino acids, or a subset thereof, e.g., the IL-21 polypeptide comprises amino acids 1-122 of SEQ ID NO:2. The corresponding polynucleotide fragments can also be used in the methods and compositions described herein. Modified polynucleotides as described above may be made by standard molecular biology techniques, including construction of appropriate desired deletion mutants, site-directed mutagenesis methods or by the polymerase chain reaction using appropriate oligonucleotide primers.

An IL-21 polypeptide can be labeled. For example, the labeled polypeptide can be used to monitor levels of the polypeptide in a subject when administered to the subject. Similarly, the labeled polypeptide can be used to monitor distribution of the polypeptide in the subject, e.g., by imaging the subject. The polypeptide can be radioactively labeled or labeled with an MRI-detectable label. Exemplary radiolabels include: ¹³¹I, ¹¹¹In, 123I, ^{99m}Tc, ³²P, ¹²⁵I, ³H, ¹⁴C, and ¹⁸⁸Rh. Exemplary MRI-detectable labels include: contrast agents such as magnetic agents, paramagnetic agents (which primarily alter T1) and ferromagnetic or superparamagnetic (which primarily alter T2 response) agents. Chelates (e.g., EDTA, DTPA and NTA chelates) can be used to attach (and reduce toxicity) of some paramagnetic substances (e.g., Fe⁺³, Mn⁺², Gd⁺³). It is also possible to attach an NMR-active atom such as an ¹⁹F atom.

In one embodiment, the IL-21 pathway agonist is a fusion protein that includes (i) a mature IL-21 polypeptide, e.g., human or murine IL-21 polypeptide, or a fragment thereof and (ii) a second moiety, e.g., a polypeptide, such as an Fc domain or a purification tag. As used herein, a “fusion protein” refers to a protein containing two or more operably associated, e.g., linked, moieties, e.g., protein moieties. Preferably, the moieties are covalently associated. The moieties can be directly associated, or connected via a spacer or linker. Additional description of IL-21 fusion proteins is available in U.S. application Ser. No. 10/806,611, filed on Mar. 22, 2004.

IL-21 Receptor

Most cytokines bind to either class I or class II cytokine receptors. Class II cytokine receptors include the receptors for IL-10 and the interferons, whereas class I cytokine receptors include the receptors for IL-2, IL-7, IL-9, IL-11-13, and IL-15; as well as the hematopoietic growth factors, leptin and growth hormone (Cosman (1993) Cytokine 5:95-106).

Human IL-21 receptor is a class I cytokine receptor that is expressed by lymphoid cells, particularly by NK, B and T cells (Parrish-Novak et al. (2000) supra). Exemplary nucleic acid sequences encoding human interleukin-21 (IL-21) and its receptor (IL-21R) are described in WO 00/53761, WO 01/85792, Parrish-Novak et al. (2000) supra, and Ozaki et al. (2000) Proc. Natl. Acad. Sci. USA 97:11439-11444, as are the corresponding amino acid sequences. IL-21 receptor shows high sequence homology to IL-2 receptor 1 chain and IL-4 receptor a chain (Ozaki et al. (2000) supra). Upon ligand binding, IL-21 receptor associates with the common gamma cytokine receptor chain (γc) that is shared by receptors for IL-2, IL-3, IL-4, IL-7, IL-9, IL-13 and IL-15 (Ozaki et al. (2000) supra; Asao et al. (2001) J. Immunol. 167:1-5).

The terms “MU-1,” “MU-1 protein,” “interleukin-21 receptor” or “IL-21R,” refer to a receptor (e.g., of mammalian, e.g., murine or human origin) which is capable of interacting with, e.g., binding to, IL-21 (e.g., of mammalian, e.g., murine or human IL-21) and having one of the following features: (i) an amino acid sequence of a naturally occurring mammalian IL-21 receptor or a fragment thereof, e.g., an amino acid sequence shown as SEQ ID NO:6 (human) or SEQ ID NO:8 (murine) or a fragment thereof (e.g., the mature region); (ii) an amino acid sequence substantially homologous to, e.g., at least 85%, 90%, 95%, 98%, 99% homologous to, an amino acid sequence shown as SEQ ID NO:6 (human) or SEQ ID NO:8 (murine) or a fragment thereof (e.g., the mature region); (iii) an amino acid sequence which is encoded by a naturally occurring mammalian IL-21 receptor nucleotide sequence (e.g., SEQ ID NO:5 (human) or SEQ ID NO:7 (murine)) or a fragment thereof (e.g., the mature region); (iv) an amino acid sequence encoded by a nucleotide sequence which is substantially homologous to, e.g., at least 85%, 90%, 95%, 98%, 99% homologous to, a nucleotide sequence shown as SEQ ID NO:5 (human) or SEQ ID NO:7 (murine) or a fragment thereof (e.g., the mature region); (v) an amino acid sequence encoded by a nucleotide sequence degenerate with respect to a naturally occurring IL-21 receptor nucleotide sequence or a fragment thereof, e.g., SEQ ID NO:5 (human) or SEQ ID NO:7 (murine) or a fragment thereof (e.g., the mature region); or (vi) an amino acid sequence, of at least 450 amino acids that is encoded a nucleotide sequence that hybridizes to one of the foregoing nucleotide sequence sequences under stringent conditions, e.g., highly stringent conditions. The mature region of the human IL-21 receptor listed in SEQ ID NO:6 is from about amino acids 20-538. Exemplary ectodomain fragments that can be used include about amino acids 20-218 or 20-232.

The following is an exemplary amino acid sequence of human IL-21 receptor (SEQ ID NO:6):

| | |
|---|---|
| MPRGWAAPLL LLLLQGGWGC PDLVCYTDYL QTVICILEMW NLHPSTLTLT WQDQYEELKD | 60 |
| EATSCSLHRS AHNATHATYT CHNDVFHFMA DDIFSVNITD QSGNYSQECG SFLLAESIKP | 120 |
| APPFNVTVTF SGQYNISWRS DYEDPAFYML KGKLQYELQY RNRGDPWAVS PRRKLISVDS | 180 |
| RSVSLLPLEF RKDSSYELQV RAGPMPGSSY QGTWSEWSDP VIFOTOSEEL KEGWNPHLLL | 240 |
| LLLLVIVFIP AFWSLKTHPL WRLWKKIWAV PSPERFFMPL YKGCSGDFKK WVGAPFTGSS | 300 |
| LELGPWSPEV PSTLEVYSCH PPRSPAKRLQ LTELQEPAEL VESDGVPKPS FWPTAQNSGG | 360 |
| SAYSEERDRP YGLVSIDTVT VLDAEGPCTW PCSCEDDGYP ALDLDAGLEP SPGLEDPLLD | 420 |
| AGTTVLSCGC VSAGSPGLGG PLGSLLDRLK PPLADGEDWA GGLPWGGRSP GGVSESEAGS | 480 |
| PLAGLDMDTF DSGFVGSDCS SPVECDFTSP GDEGPPRSYL RQWVVIPPPL SSPGPQAS | 538 |

The following is an exemplary amino acid sequence of murine IL-21 receptor (SEQ ID NO:8):

| | |
|---|---|
| MPRGPVAALL LLILHGAWSC LDLTCYTDYL WTITCVLETR SPNPSILSLT WQDEYEELQD | 60 |
| QETFCSLHRS GHNTTHIWYT CHMRLSQFLS DEVFIVNVTD QSGNNSQECG SFVLAESIKP | 120 |
| APPLNVTVAF SGRYDISWDS AYDEPSNYVL RGKLQYELQY RNLRDPYAVR PVTKLISVDS | 180 |
| RNVSLLPEEF HKDSSYQLQV RAAPQPGTSF RGTWSEWSDP VIFQTQAGEP EAGWDPHMLL | 240 |
| LLAVLIIVLV FMGLKIHLPW RLWKKIWAPV PTPESFFQPL YREHSGNFKK WVNTPFTASS | 300 |
| IELVPQSSTT TSALHLSLYP AKEKKFPGLP GLEEQLECDG MSEPGHWCII PLAAGQAVSA | 360 |
| YSEERDRPYG LVSIDTVTVG DAEGLCVWPC SCEDDGYPAM NLDAGRESGP NSEDLLLVTD | 420 |
| PAFLSCGCVS GSGLRLGGSP GSLLDRLRLS FAKEGDWTAD PTWRTGSPGG GSESEAGSPP | 480 |
| GLDMDTFDSG FAGSDCGSPV ETDEGPPRSY LRQWVVRTPP PVDSGAQSS | 529 |

An exemplary IL-21R/MU-1 cDNA was deposited with the American Type Culture Collection on Mar. 10, 1998, as accession number ATCC 98687. An IL-21 receptor may have additional conservative or non-essential amino acid substitutions, which do not have a substantial effect on their functions, e.g., a substitution described herein.

IL-21 receptor is a class I cytokine family receptor, also known as NILR (WO 01/85792; Parrish-Novak et al. (2000) Nature 408:57-63; Ozaki et al. (2000) Proc. Natl. Acad. Sci. USA 97:11439-11444). IL-21 receptor is expressed in lymphoid tissue. IL-21 receptor is homologous to the shared β chain of the IL-2 and IL-15 receptors, and IL-4 receptor α chain (Ozaki et al. (2000) supra). Upon ligand binding, IL-21R/MU-1 is capable of interacting with a common γ cytokine receptor chain (γc) (Asao et al. (2001) J. Immunol. 167:1-5), and inducing the phosphorylation of STAT1 and STAT3 (Asao et al. (2001) J. Immunol. 167:1-5 or STAT5 (Ozaki et al. (2000). The term “IL-21 receptor complex” refers to a protein complex that includes the IL-21 receptor and at least one additional cell-associated protein component, e.g., the β chain or common γ cytokine receptor chain. Typically, the IL-21 receptor complex includes the IL-21 receptor, the β chain and the common γ cytokine receptor chain.

The phrase “a biological activity of” a IL-21 receptor refers to one or more of the biological activities of the corresponding mature IL-21 receptor, including, but not limited to, (1) interacting with, e.g., binding to, an IL-21 polypeptide (e.g., a human IL-21 polypeptide); (2) associating with signal transduction molecules, e.g., γc, jak1; (3) stimulating phosphorylation and/or activation of STAT proteins, e.g., STAT5 and/or STAT3; and/or (4) modulating, e.g., stimulating or decreasing, proliferation, differentiation, agonist cell function, cytolytic activity, cytokine secretion, and/or survival of immune cells, e.g., T cells (CD8+, CD4+ T cells), NK cells, B cells, macrophages and megakaryocytes).

Additional Exemplary IL-21 Pathway Agonists

In one embodiment, an IL-21 pathway agonist is an agent that interacts with IL-21 receptor, but is other than an IL-21 polypeptide. For example, the agent can be an immunoglobulin, e.g., a full length antibody or antibody fragment, that interacts with an IL-21 receptor and that activates IL-21 pathway signaling activity, e.g., by agonizing the receptor.

In one embodiment, an IL-21 pathway agonist is an agent that interacts with IL-21 receptor and another receptor subunit, e.g., γc. For example, the agent can be a protein that interacts with IL-21 receptor and another receptor subunit, e.g., γc. The protein can be, e.g., a bispecific antibody that includes one antigen binding site that interacts with IL-21 receptor and another antigen binding site that interacts with γc. Binding of such a protein can be used to crosslink and agonize the receptor, e.g., activate or increase STAT3 or STAT5 signaling.

In one embodiment, an IL-21 pathway agonist is an agent (e.g., an immunoglobulin) that stabilizes an IL-21/IL-21R interaction, e.g., by binding one or both of IL-21 and IL-21 receptor.

An IL-21 pathway agonist can be identified, e.g., by screening protein libraries, chemical libraries, engineering and design, or evaluating a test compound, e.g., for binding and/or activation of an IL-21 receptor using procedures known in the art. Binding assays using a desired binding protein, immobilized or not, are known in the art and may be used for this purpose using the IL-21 receptor protein as described herein. Purified cell based or protein based (cell free) screening assays may be used to identify such agonists. For example, IL-21 receptor protein may be immobilized in purified form on a carrier and binding or potential ligands to purified IL-21 receptor protein may be measured. Cell-based assays for evaluating IL-21 receptor activity and STAT (e.g., STAT1, STAT3 or STAT5) signaling are known. Examples are described herein and in Asao et al. (2001) J. Immunol. 167:1-5, Ozaki et al. (2000) supra, U.S. Ser. No. 10/806,611, filed on Mar. 22, 2004, and U.S. 2003-0108549.

IL-21 Pathway Antagonists

In another aspect of the invention, an IL-21 pathway antagonist can be used to increase IgE production and/or decrease IgG production. An “IL-21 pathway antagonist” is an agent that decrease IL-21 pathway signaling. For example, such an agent can decrease IL-21 receptor activity.

Exemplary IL-21 pathway antagonists include agents agent that bind to IL-21 or to IL-21 receptor. An antibody that binds to IL-21 can prevent IL-21 from interacting with the IL-21 receptor or from activating the IL-21 receptor. Another agent that binds to IL-21 and can function as a pathway antagonist is a soluble form of the IL-21 receptor, e.g., the IL-21 receptor ectodomain, or other region of the receptor sufficient to interact with IL-21. In one embodiment, the agent is an Fc fusion protein that includes an Fc domain and region of the receptor sufficient to interact with IL-21. An antibody that binds to the IL-21 receptor can also function as a pathway antagonist. Such an antibody may prevent IL-21 from interact with or activating the receptor.

Still other pathway antagonists include small molecule inhibitors of cytoplasmic signaling components, e.g., small molecule inhibitors of STAT3 and STAT5. Nucleic acid molecules that can function as pathway antagonists are described below.

Immunoglobulins

Immunoglobulin molecules can be used to modulate IL-21 pathway activity. For example, one class of immunoglobulin molecules includes molecules that bind to the IL-21 receptor and increases IL-21 pathway activity. Another exemplary class of immunoglobulin molecules includes molecules that bind to the IL-21 polypeptide or the IL-21 receptor and decrease IL-21 pathway activity.

A typical immunoglobulin is an antibody. As used herein, the term “antibody” refers to a protein comprising at least one, and preferably two, heavy (H) chain variable domains (abbreviated herein as VH), and at least one and preferably two light (L) chain variable domains (abbreviated herein as VL). The VH and VL domains can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDRs and four FRs, arranged from amino-terminus to carboxy-terminus in the following order: FR1, CDR1, FR2, CDR2, FR3, CDR3, FR4. Camelid antibodies can include a single variable immunoglobulin domain.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable domain of the heavy and light chains contains a binding domain that interacts with an antigen. The constant regions of the antibodies typically mediate the binding of the antibody to host tissues or factors, including various cells of the immune system (e.g., agonist cells) and the first component (C1q) of the classical complement system.

As used herein, the term “immunoglobulin” refers to a protein that includes one or more polypeptides that have a domain that forms an immunoglobulin fold. An immunoglobulin domain is roughly a cylinder (about 4×2.5×2.5 nm) with two extended protein layers: one layer contains three strands of polypeptide chain and the other contains four. In each layer the adjacent strands are antiparallel and form a β-sheet. The two layers are roughly parallel and are often connected by a single intrachain disulfide bond.

An immunoglobulin can include a region encoded by an immunoglobulin gene. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin genes and gene segments. Full-length immunoglobulin “light chains” (about 25 Kd or 214 amino acids) are encoded by a variable region gene at the NH2-terminus (about 110 amino acids) and a kappa or lambda constant region gene at the COOH—terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids). As used herein, “isotype” refers to the antibody class (e.g., IgM, IgG1, IgG2, IgG3, IgG4) that is encoded by heavy chain constant region genes.

The term “antigen-binding fragment” of an antibody (or simply “antibody portion,” or “fragment”), as used herein, refers to one or more fragments of a full-length antibody that retain the ability to specifically bind to an antigen (e.g., IL-21 receptor). Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. An “substantially human” immunoglobulin variable domain is an immunoglobulin variable domain that includes a sufficient number of human framework amino acid positions such that the immunoglobulin variable domain does not elicit an immunogenic response in a normal human. An “substantially human” antibody is an antibody that includes a sufficient number of human amino acid positions such that the antibody does not elicit an immunogenic response in a normal human. Human and substantially human immunoglobulin variable domains and antibodies can be used.

IL-21 polypeptide and IL-21 receptor proteins may be used to immunize animals (e.g., non-human animals and non-human animals include human immunoglobulin genes) to obtain polyclonal and monoclonal antibodies which specifically react with the IL-21 polypeptide or IL-21 receptor protein and which may activate an IL-21 receptor. Such antibodies may be obtained using the entire mature protein as an immunogen, or by using fragments of IL-21/IL-21R (e.g., soluble fragments and small peptides). The peptide immunogens additionally may contain a cysteine residue at the carboxyl terminus, and are conjugated to a hapten such as keyhole limpet hemocyanin (KLH). Additional peptide immunogens may be generated by replacing tyrosine residues with sulfated tyrosine residues. Methods for synthesizing such peptides are known in the art, for example, as in R. P. Merrifield, J. Amer. Chem. Soc. 85, 2149-2154 (1963); J. L. Krstenansky, et al., FEBS Lett. 211, 10 (1987).

Human monoclonal antibodies (mAbs) directed against IL-21 or IL-21 receptor can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., WO 91/00906, WO 91/10741; WO 92/03918; WO 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies (for descriptions of combinatorial antibody display see e.g., Sastry et al. 1989 PNAS 86:5728; Huse et al. 1989 Science 246:1275; and Orlandi et al. 1989 PNAS 86:3833). After immunizing an animal with an immunogen as described above, the antibody repertoire of the resulting B-cell pool is cloned. Methods are generally known for obtaining the DNA sequence of the variable domains of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable domains from a number of murine antibodies (Larrick et al., 1991, Biotechniques 11:152-156). A similar strategy can also been used to amplify human heavy and light chain variable domains from human antibodies (Larrick et al., 1991, Methods: Companion to Methods in Enzymology 2:106-110).

Chimeric antibodies, including chimeric immunoglobulin chains, can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

An antibody or an immunoglobulin chain can be humanized by methods known in the art. Humanized antibodies, including humanized immunoglobulin chains, can be generated by replacing sequences of the Fv variable domain which are not directly involved in antigen binding with equivalent sequences from human Fv variable domains. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable domains from at least one of a heavy or light chain. Sources of such nucleic acid are well known to those skilled in the art and, for example, may be obtained from a hybridoma producing an antibody against a predetermined target. The recombinant DNA encoding the humanized antibody, or fragment thereof, can then be cloned into an appropriate expression vector.

Humanized or CDR-grafted antibody molecules or immunoglobulins can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies (UK Patent Application GB 2188638A, filed on Mar. 26, 1987; Winter U.S. Pat. No. 5,225,539), the contents of which is expressly incorporated by reference. All of the CDRs of a particular human antibody may be replaced with at least a portion of a non-human CDR or only some of the CDRs may be replaced with non-human CDRs. It is only necessary to replace the number of CDRs required for binding of the humanized antibody to a predetermined antigen.

In some implementations, monoclonal, chimeric and humanized antibodies can be modified by, e.g., deleting, adding, or substituting other portions of the antibody, e.g., the constant region. For example, an antibody can be modified as follows: (i) by deleting the constant region; (ii) by replacing the constant region with another constant region, e.g., a constant region meant to increase half-life, stability or affinity of the antibody, or a constant region from another species or antibody class; or (iii) by modifying one or more amino acids in the constant region to alter, for example, the number of glycosylation sites, agonist cell function, Fc receptor (FcR) binding, complement fixation, among others.

Methods for altering antibody constant regions are known. Antibodies with altered function, e.g. altered affinity for an agonist ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260). Similar type of alterations could be described which if applied to the murine, or other species immunoglobulin would reduce or eliminate these functions.

Nucleic Acid Antagonists of the IL-21 Pathway

In certain implementations, nucleic acid antagonists are used to decrease IL-21 pathway activity, e.g., to decrease IgG production. In one embodiment, the nucleic acid antagonist is an siRNA that targets mRNA encoding an IL-21 polypeptide or an IL-21 receptor, or other positively acting IL-21 pathway component can be used to decrease IL-21 pathway activity. Other types of antagonistic nucleic acids can also be used, e.g., a nucleic acid aptamer, a dsRNA, a ribozyme, a triple-helix former, or an antisense nucleic acid.

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically the siRNA sequences are exactly complementary to the target mRNA. dsRNAs and siRNAs in particular can be used to silence gene expression in mammalian cells (e.g., human cells). See, e.g., Clemens, J. C. et al. (2000) Proc. Natl. Sci. USA 97, 6499-6503; Billy, E. et al. (2001) Proc. Natl. Sci. USA 98, 14428-14433; Elbashir et al. (2001) Nature. 411(6836):494-8; Yang, D. et al. (2002) Proc. Natl. Acad. Sci. USA 99, 9942-9947, U.S. 20030166282, 20030143204, 20040038278, and 20030224432.

Descriptions of other types of nucleic acid agents are also available. See, e.g., U.S. Pat. No. 4,987,071; U.S. Pat. No. 5,116,742; U.S. Pat. No. 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher, L. J. (1992) Bioassays 14:807-15.

Recombinant Protein Production

The nucleic acids encoding proteins that function as agents for the methods described herein may be operably linked to an expression control sequence in a vector (such as the pMT2 or pED expression vectors disclosed in Kaufman et al., Nucleic Acids Res. 19, 4485-4490 (1991)), in order to produce the protein recombinantly. Many suitable expression control sequences are known. General methods of expressing recombinant proteins are also known and are exemplified in R. Kaufman, Methods in Enzymology 185, 537-566 (1990), Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989). As defined herein “operably linked” means enzymatically or chemically ligated to form a covalent bond between a particular polynucleotide encoding a protein of interest and the expression control sequence, in such a way that the protein of interest (e.g., IL-21 or another IL-21 pathway agonist) is expressed by a host cell which has been transformed (transfected) with the ligated polynucleotide/expression control sequence.

The term “vector,” as used herein, refers to a nucleic acid molecule capable of transporting, or sustaining maintenance or replication of, another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or “expression vectors.” Exemplary viral vectors include replication defective retroviruses, adenoviruses and adeno-associated viruses.

The term “regulatory sequence” is intended to includes promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). The selection of regulatory sequences may depend on such factors as the choice of the host cell to be transformed, the level of expression of protein desired, etc. Exemplary regulatory sequences for mammalian host cell expression include viral elements that direct high levels of protein expression in mammalian cells, such as promoters and/or enhancers derived from FF-1a promoter and BGH poly A, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further exemplary descriptions of viral regulatory elements, and sequences thereof, see e.g., U.S. Pat. No. 5,168,062, U.S. Pat. No. 4,510,245, and U.S. Pat. No. 4,968,615.

The recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr− host cells with methotrexate selection/amplification) and the neo gene (for G418 selection). A number of types of cells may act as suitable host cells for expression of a protein therapeutic. Any cell type capable of expressing the protein therapeutic may be used. Exemplary mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK, Rat2, BaF3, 32D, FDCP-1, PC12, M1x or C2C12 cells.

A protein therapeutic may be produced by operably linking a polynucleotide encoding such a protein to suitable control sequences in one or more insect expression vectors, and employing an insect expression system. Materials and methods for baculovirus/insect cell expression systems are commercially available, e.g., in kit form from, e.g., Invitrogen, San Diego, Calif. U.S.A. (the MAXBAC® kit), e.g., as described in Summers and Smith, Texas Agricultural Experiment Station Bulletin No. 1555 (1987). Soluble forms of the IL-21 receptor protein may also be produced in insect cells using appropriate isolated polynucleotides, e.g., forms in which the region encoding one or more, or sufficient segments, of the transmembrane domain and the cytoplasmic domain are removed.

A protein therapeutic may be produced in lower eukaryotes such as yeast or in prokaryotes such as bacteria. Suitable yeast strains include Saccharomyces cerevisiae, Schizosaccharomyces pombe, Kluyveromyces strains, Pichia, Candida, or any yeast strain capable of expressing heterologous proteins. Suitable bacterial strains include Escherichia coli, Bacillus subtilis, Salmonella typhimurium, or any bacterial strain capable of expressing heterologous proteins.

In one embodiment, an IL-21 polypeptide is produced in a bacterial cell without a signal sequence (e.g., without either a prokaryotic or eukaryotic signal sequence). Expression in bacteria may result in formation of inclusion bodies incorporating the recombinant protein. Thus, refolding of the recombinant protein may be required in order to produce active or more active material. Several methods for obtaining correctly folded heterologous proteins from bacterial inclusion bodies are known in the art. These methods generally involve solubilizing the protein from the inclusion bodies, then denaturing the protein completely using a chaotropic agent.

When cysteine residues are present in the primary amino acid sequence of the protein, the protein can be refolded in an environment which facilitates correct formation of disulfide bonds (e.g., a redox system). General methods of refolding are disclosed in Kohno, Meth. Enzym., 185:187-195 (1990). EP 0433225 and U.S. Pat. No. 5,399,677. Asano et al. (2002) FEBS Lett. 528(1-3):70-6 describes an exemplary method for refolding IL-21 produced in bacterial cells. For example, rIL-21 (recombinant IL-21) is expressed as insoluble inclusion bodies in E. coli, then solubilized (e.g., using a denaturant) and refolded by using a modified dialysis method in which redox reagents are introduced.

The IL-21 pathway agonist protein or fusion protein thereof may also be expressed as a product of transgenic animals, e.g., as a component of the milk of transgenic cows, goats, pigs, or sheep which are characterized by somatic or germ cells containing a polynucleotide sequence encoding the IL-21 pathway agonist protein or fusion protein thereof.

Treatments

In one aspect of the invention, an IL-21 pathway agonist is used to treat or prevent an atopic disorder.

As used herein, the term “treat” or “treatment” is defined as the application or administration of a composition to a subject (e.g., a human subject, e.g., a patient or person at risk for a disorder, e.g., an atopic disorder). In certain implementations treatments can include application or administration of the agent to an isolated tissue or cell, e.g., cell line, from a subject, e.g., a patient. Generally, a treatment is provided to a subject who has a disorder (e.g., a disorder as described herein), a symptom of a disorder, an elevated risk for a disorder, or a predisposition for a disorder, with a purpose to cure, heal, alleviate, relieve, alter, remedy, ameliorate, improve or affect the disorder, the symptoms of the disorder or the predisposition toward the disorder. Treatments can include administering or applying the composition alone or in combination with, a second agent. The term “in combination” in this context means that different agents are given substantially contemporaneously, either simultaneously or sequentially. If given sequentially, at the onset of administration of the second compound, the first of the two agents is preferably still detectable at effective concentrations at the site of treatment.

“Treating a cell” refers to contacting an agent to a cell, e.g., an immune cell, for example, to change a behavior or state of the cell. In one embodiment, treating a cell with a modulator of the IL-21 pathway can be used to modulate (e.g., increase or decrease) production of IgG or IgE.

As used herein, an amount of an agent effective to treat a disorder, or a “therapeutically effective amount” refers to an amount of the compound which is effective, upon single or multiple dose administration to a subject, in treating a subject, e.g., curing, alleviating, relieving or improving at least one symptom of a disorder in a subject to a degree beyond that expected in the absence of such treatment. For example, the disorder can be an atopic disorder, e.g., a an atopic disorder described herein.

A “locally effective amount” refers to the amount (e.g., concentration) of the compound which is effective at detectably modulating cells in a tissue, e.g., in a region of an atopic disorder, to modulate cell activity. Evidence of modulation can include, e.g., modulation of IgG or IgE production.

As used herein, an amount of an agent “effective to prevent a disorder,” or “a prophylactically effective amount” of the compound refers to an amount of the agent which is effective, upon single- or multiple-dose administration to the subject, in preventing or delaying the occurrence of the onset or recurrence of a disorder, e.g., an atopic disorder.

A pharmaceutical composition may include a “therapeutically effective amount” or a “prophylactically effective amount” of an agent described herein, e.g., an IL-21 polypeptide, an antibody, or a form of an IL-21 receptor. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the compound to elicit a desired response in the individual. A therapeutically effective amount is also one in which any toxic or detrimental effects of the composition is outweighed by the therapeutically beneficial effects. A “therapeutically effective dosage” preferably modulates a measurable parameter, e.g., immunoglobulin production or a measurable symptom of an atopic disorder relative to untreated subjects, e.g., to a statistically significant degree. The ability of a compound to inhibit a measurable parameter can be evaluated in an animal model system predictive of efficacy in a human disorder, using in vitro assays, e.g., an assay described herein, or using appropriate human trials.

Particular effects mediated by an IL-21 pathway agonist or antagonist may show a difference that is statistically significant (e.g., P value<0.05 or 0.02). Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02. The terms “induce”, “inhibit”, “potentiate”, “elevate”, “increase”, “decrease” or the like, e.g., which denote distinguishable qualitative or quantitative differences between two states, and may refer to a difference, e.g., a statistically significant difference (e.g., P value<0.05 or 0.02), between the two states.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is possible to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

An exemplary, non-limiting range for a therapeutically or prophylactically effective amount of an agent described herein is 0.1-20 mg/kg, more preferably 1-10 mg/kg. The agent can be administered by intravenous infusion at a rate of less than 20, 10, 5, or 1 mg/min to reach a dose of about 1 to 50 mg/m² or about 5 to 20 mg/m². Dosage values may vary with the type and severity of the condition to be alleviated. For any individual subject, specific dosage regimens can be adjusted over time according to the individual need and the professional judgment of the person administering or supervising the administration of the compositions. Accordingly, the dosage ranges set forth herein are only exemplary.

As used herein, the term “subject” is intended to include human and non-human animals. The term “non-human animals” of the invention includes all vertebrates, e.g., non-mammals (such as chickens, amphibians, reptiles) and mammals, such as non-human primates, mice, sheep, dogs, cows, pigs, etc.

Some exemplary methods of administering compounds are described in “Pharmaceutical Compositions.” Pharmaceutical compositions can be also administered using a medical device. For example, in one embodiment, a pharmaceutical composition of the invention can be administered with a needle-less hypodermic injection device, such as the devices disclosed in U.S. Pat. No. 5,399,163, 5,383,851, 5,312,335, 5,064,413, 4,941,880, 4,790,824, or 4,596,556. Examples of well-known implants and modules that can be used include: U.S. Pat. No. 4,487,603, which discloses an implantable micro-infusion pump for dispensing medication at a controlled rate; U.S. Pat. No. 4,486,194, which discloses a therapeutic device for administering agents through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

In one embodiment, the agent is formulated for respiratory or mucosal delivery, e.g., using a medical device, e.g., an inhaler. See, e.g., U.S. Pat. No. 6,102,035 (a powder inhaler) and U.S. Pat. No. 6,012,454 (a dry powder inhaler). In one embodiment, the inhaler is a metered dose inhaler. Three common systems used to deliver drugs locally to the pulmonary air passages include dry powder inhalers (DPIs), metered dose inhalers (MDIs) and nebulizers. MDIs, the most popular method of inhalation administration, may be used to deliver medicaments in a solubilized form or as a dispersion. Typically MDIs comprise a Freon or other relatively high vapor pressure propellant that forces aerosolized medication into the respiratory tract upon activation of the device. Unlike MDIs, DPIs generally rely entirely on the inspiratory efforts of the patient to introduce a medicament in a dry powder form to the lungs. Nebulizers form a medicament aerosol to be inhaled by imparting energy to a liquid solution. Direct pulmonary delivery of drugs during liquid ventilation or pulmonary lavage using a fluorochemical medium is also possible.

In one embodiment, an IL-21 pathway agonist is administered topically. “Topical administration” refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. The term also encompasses transdermal routes of administration. Topical modes of administration typically include penetration of the skin's permeability barrier and efficient delivery to the target tissue or stratum. Topical administration can be used as a means to penetrate the epidermis and dermis and achieve local or systemic delivery of the composition. Topical administration can also be used as a means to selectively deliver an IL-21 pathway agonist to the skin (e.g., the epidermis or dermis) of a subject, or to specific strata thereof, or to an underlying tissue. The term “skin,” as used herein, refers to the epidermis and/or dermis of an animal.

Several factors determine the permeability of the skin to administered agents. These factors include the characteristics of the treated skin, the characteristics of the delivery agent, interactions between both the drug and delivery agent and the drug and skin, the dosage of the drug applied, the form of treatment, and the post treatment regimen. To selectively target the epidermis and dermis, it is sometimes possible to formulate a composition that comprises one or more penetration enhancers that will enable penetration of the drug to a preselected stratum.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose position and retention at the site of administration (Lee et al., Critical Reviews in Therapeutic Drug Carrier Systems, 1991, p. 168) may be useful methods for enhancing the transport of topically applied agents across skin and mucosal sites.

Pharmaceutical Compositions

IL-21 pathway agonists may be used as a pharmaceutical composition when combined with a pharmaceutically acceptable carrier. Such a composition may contain, in addition to the IL-21 pathway agonists and carrier, various diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier typically depend on the route of administration.

The pharmaceutical composition may further contain other anti-inflammatory agents as described in more detail below. Such additional factors and/or agents may be included in the pharmaceutical composition to produce a synergistic effect with an IL-21 pathway agonists, or to minimize side effects caused by the IL-21 pathway agonists. Conversely IL-21 pathway agonists may be included in formulations of the particular anti-inflammatory agent to minimize side effects of the anti-inflammatory agent.

The pharmaceutical composition may be in the form of a liposome in which IL-21 pathway agonists is combined, in addition to other pharmaceutically acceptable carriers, with amphipathic agents such as lipids which exist in aggregated form as micelles, insoluble monolayers, liquid crystals, or lamellar layers which in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

In practicing the method of treatment or use, a therapeutically effective amount of an IL-21 pathway agonist or antagonist is administered to a subject, e.g., mammal (e.g., a human). An IL-21 pathway agonists may be administered either alone or in combination with other therapies such as other treatments for atopic disorders. When co-administered with one or more agents, the IL-21 pathway agonist may be administered either simultaneously with the second agent, or sequentially. If administered sequentially, the attending physician can decide on the appropriate sequence of administering an IL-21 pathway agonist in combination with other agents.

Exemplary additional agents for use in treating atopic disorders include: other immunomodulators (e.g., tacrolimus ointment (PROTOPIC™) and pimecromlimus cream (ELIDEL™)), corticosteroids (topical and systemic), antihistamines, immunosuppressants (e.g., cyclosporine, methotrexate or azathioprine). Exemplary additional agents for use in treating an allergic disorder include: CLARITIN® (loratadine), diphenhydramine, and other anti-histamines, and ketotifen fumarate.

Administration of an IL-21 pathway agonist can be carried out in a variety of ways, including, for example, oral ingestion, intracranial, inhalation, or cutaneous, subcutaneous, or intravenous injection or administration. For example, the composition can be delivered as an epidural or otherwise, e.g., to cerebrospinal fluid.

To orally administer a therapeutically effective amount of an IL-21 pathway agonist, the agent can be in the form of a tablet, capsule, powder, solution or elixir. When administered in tablet form, the pharmaceutical composition may additionally contain a solid carrier such as a gelatin or an adjuvant. The tablet, capsule, and powder contain from about 5 to 95% of the agent or from about 25 to 90% of the agent. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the agent, and preferably from about 1 to 50% the agent.

To administer a therapeutically effective amount of an IL-21 pathway agonist, e.g., by intravenous, cutaneous or subcutaneous injection, the agent can be in the form of a pyrogen-free, parenterally acceptable aqueous solution. The preparation of such parenterally acceptable protein solutions, having due regard to pH, isotonicity, stability, and the like, is within the skill in the art. An exemplary pharmaceutical composition for intravenous, cutaneous, or subcutaneous injection can contain, in addition to the agent an isotonic vehicle such as sodium chloride injection, Ringer's injection, dextrose injection, dextrose and sodium chloride injection, lactated Ringer's injection, or other vehicle as known in the art. The pharmaceutical composition of the present invention may also contain stabilizers, preservatives, buffers, antioxidants, or other additive known to those of skill in the art.

The amount of an IL-21 pathway agonist in the pharmaceutical composition of the present invention can depend upon the nature and severity of the condition being treated, and on the nature of prior treatments that the patient has undergone. The attending physician can decide the amount of agonist with which to treat each individual patient. Initially, for example, the attending physician can administer low doses of the agent and observe the patient's response. Larger doses of the agent may be administered until the optimal therapeutic effect is obtained for the patient, and at that point the dosage is not generally increased further, or by monitoring immunoglobulin levels (e.g., IgG or IgE levels) or one or more symptoms. Exemplary pharmaceutical compositions may contain about 0.1 μg to about 100 mg IL-21 pathway agonist per kg body weight. For example, useful dosages can include between about 10 μg-1 mg, 0.1-5 mg, and 3-50 mg of IL-21 pathway agonist per kg body weight. Useful dosages of IL-21 can further include between about 5 μg-1 mg, 0.1-5 mg, and 3-20 mg of IL-21 pathway agonist per kg body weight.

The duration of intravenous therapy using the pharmaceutical composition can vary, depending on the severity of the disease being treated and the condition and potential idiosyncratic response of each individual patient. The duration of each application of the IL-21 pathway agonist can be, e.g., in the range of 12 to 24 hours of continuous intravenous administration. The attending physician can decide on the appropriate duration of intravenous therapy using the pharmaceutical composition of the present invention.

In one embodiment, the IL-21 pathway agonist is formulated as a microparticle or other sustained-release formulation. Microparticles can be produced by spray-drying, but may also be produced by other methods including lyophilization, evaporation, fluid bed drying, vacuum drying, or a combination of these techniques. Controlled or sustained release can be achieved by disposing the agonist within a structure or substance which impedes its release. For example, the agonist can be disposed within a porous matrix or in an erodable matrix, either of which allow release of the agonist over a period of time.

In one embodiment, a mixed micellar formulation that includes an IL-21 pathway agonist is used to deliver the agent through transdermal membranes. The formulation may be prepared, for example, by mixing an aqueous solution of the IL-21 pathway agonist, and a micelle forming compounds, and optionally, an alkali metal, e.g., C₈ to C₂₂ alkyl sulphate. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing is preferred in order to provide smaller size micelles. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

IL-21 pathway antagonists may be formulated and prepared as pharmaceutical composition combined with a pharmaceutically acceptable carrier in a manner similar to that described for IL-21 pathway agonists.

With respect to IL-21 pathway agonists and antagonists that are proteins, the disease or disorder can also be treated or prevented by administration or use of polynucleotides encoding such proteins (such as, for example, in gene therapies or vectors suitable for introduction of DNA). The polynucleotides that encode an IL-21 pathway agonist (e.g., an IL-21 polypeptide) can be inserted into vectors and used as gene therapy vectors. Gene therapy vectors can be delivered to a subject by, for example, intravenous injection, local administration (see U.S. Pat. No. 5,328,470), injection (e.g., U.S. 20040030250 or 20030212022) or stereotactic injection (e.g., Chen et al. Proc. Natl. Acad. Sci. USA 91:3054-3057, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Kits

An IL-21 pathway agonist described herein, e.g., an IL-21 polypeptide or an antibody that binds to a IL-21 receptor, can be provided in a kit. The kit includes (a) IL-21 pathway agonist, e.g., a composition that includes IL-21 pathway agonist, and, optionally (b) informational material. The informational material can be descriptive, instructional, marketing or other material that relates to the methods described herein and/or the use of IL-21 pathway agonist for the methods described herein.

The informational material of the kits is not limited in its form. In one embodiment, the informational material can include information about production of the compound (i.e., the IL-21 pathway agonist), molecular weight of the compound, concentration, date of expiration, batch or production site information, and so forth. In one embodiment, the informational material relates to administration of the compound for treating or prevent an atopic disorder.

In one embodiment, the informational material can include instructions to administer IL-21 pathway agonist in a suitable manner to perform the methods described herein, e.g., in a suitable dose, dosage form, or mode of administration (e.g., a dose, dosage form, or mode of administration described herein). Exemplary doses, dosage forms, or modes of administration are about 10 μg-1 mg, 0.1-5 mg, and 3-50 mg of IL-21 polypeptide per kg body weight. In another embodiment, the informational material can include instructions to administer IL-21 pathway agonist to a suitable subject, e.g., a human, e.g., a human having, or at risk for, an atopic disorder. For example, the material can include instructions to administer IL-21 pathway agonist to ameliorate at least one system of the atopic disorder, e.g., asthma, atopic dermatitis, or allergic rhinitis.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as computer readable material, video recording, or audio recording. In another embodiment, the informational material of the kit is contact information, e.g., a physical address, email address, website, or telephone number, where a user of the kit can obtain substantive information about IL-21 pathway agonist and/or its use in the methods described herein. Of course, the informational material can also be provided in any combination of formats.

In addition to IL-21 pathway agonist, the composition of the kit can include other ingredients, such as a solvent or buffer, a stabilizer, a preservative, a flavoring agent (e.g., a bitter antagonist or a sweetener), a fragrance or other cosmetic ingredient, and/or a second agent for treating a condition or disorder described herein, e.g., an atopic disorder, e.g., asthma, atopic dermatitis, or allergic rhinitis. Alternatively, the other ingredients can be included in the kit, but in different compositions or containers than IL-21 pathway agonist. In such embodiments, the kit can include instructions for admixing IL-21 pathway agonist and the other ingredients, or for using IL-21 pathway agonist together with the other ingredients.

IL-21 pathway agonist can be provided in any form, e.g., liquid, dried or lyophilized form. It is preferred that IL-21 pathway agonist be substantially pure and/or sterile. When IL-21 pathway agonist is provided in a liquid solution, the liquid solution preferably is an aqueous solution, with a sterile aqueous solution being preferred. When IL-21 pathway agonist is provided as a dried form, reconstitution generally is by the addition of a suitable solvent. The solvent, e.g., sterile water or buffer, can optionally be provided in the kit.

The kit can include one or more containers for the composition containing IL-21 pathway agonist. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of IL-21 pathway agonist. For example, the kit includes a plurality of syringes, ampules, foil packets, or blister packs, each containing a single unit dose of IL-21 pathway agonist. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is an inhaler or an implantable pump.

Atopic Disorders and Symptoms Thereof

“Atopic” refers to a group of diseases where there is often an inherited tendency to develop an allergic reaction. Examples of atopic disorders include allergy, allergic rhinitis, atopic dermatitis, asthma and hay fever.

Asthma is a phenotypically heterogeneous disorder associated with intermittent respiratory symptoms such as, e.g., bronchial hyperresponsiveness and reversible airflow obstruction. Immunohistopathologic features of asthma include, e.g., denudation of airway epithelium, collagen deposition beneath the basement membrane; edema; mast cell activation; and inflammatory cell infiltration (e.g., by neutrophils, eosinophils, and lymphocytes). Airway inflammation can further contribute to airway hyperresponsiveness, airflow limitation, acute bronchoconstriction, mucus plug formation, airway wall remodeling, and other respiratory symptoms. An IL-21 pathway agonist can be administered to ameliorate one or more of these symptoms.

Symptoms of allergic rhinitis (hay fever) include itchy, runny, sneezing, or stuffy noses, and itchy eyes. An IL-21 pathway agonist can be administered to ameliorate one or more of these symptoms.

Atopic dermatitis is a chronic (long-lasting) disease that affects the skin. Information about atopic dermatitis is available, e.g., from NIH Publication No. 03-4272. In atopic dermatitis, the skin can become extremely itchy, leading to redness, swelling, cracking, weeping clear fluid, and finally, crusting and scaling. In many cases, there are periods of time when the disease is worse (called exacerbations or flares) followed by periods when the skin improves or clears up entirely (called remissions).

Atopic dermatitis is often referred to as “eczema,” which is a general term for the several types of inflammation of the skin. Atopic dermatitis is the most common of the many types of eczema. Examples of atopic dermatitis include: allergic contact eczema (dermatitis: a red, itchy, weepy reaction where the skin has come into contact with a substance that the immune system recognizes as foreign, such as poison ivy or certain preservatives in creams and lotions); contact eczema (a localized reaction that includes redness, itching, and burning where the skin has come into contact with an allergen (an allergy-causing substance) or with an irritant such as an acid, a cleaning agent, or other chemical); dyshidrotic eczema (irritation of the skin on the palms of hands and soles of the feet characterized by clear, deep blisters that itch and burn); neurodermatitis (scaly patches of the skin on the head, lower legs, wrists, or forearms caused by a localized itch (such as an insect bite) that become intensely irritated when scratched); nummular eczema (coin-shaped patches of irritated skin—most common on the arms, back, buttocks, and lower legs—that may be crusted, scaling, and extremely itchy); seborrheic eczema (yellowish, oily, scaly patches of skin on the scalp, face, and occasionally other parts of the body). Additional particular symptoms include stasis dermatitis, atopic pleat (Dennie-Morgan fold), cheilitis, hyperlinear palms, hyperpigmented eyelids: eyelids that have become darker in color from inflammation or hay fever, ichthyosis, keratosis pilaris, lichenification, papules, and urticaria. An IL-21 pathway agonist can be administered to ameliorate one or more of these symptoms.

Assays for Evaluating Candidate Agents

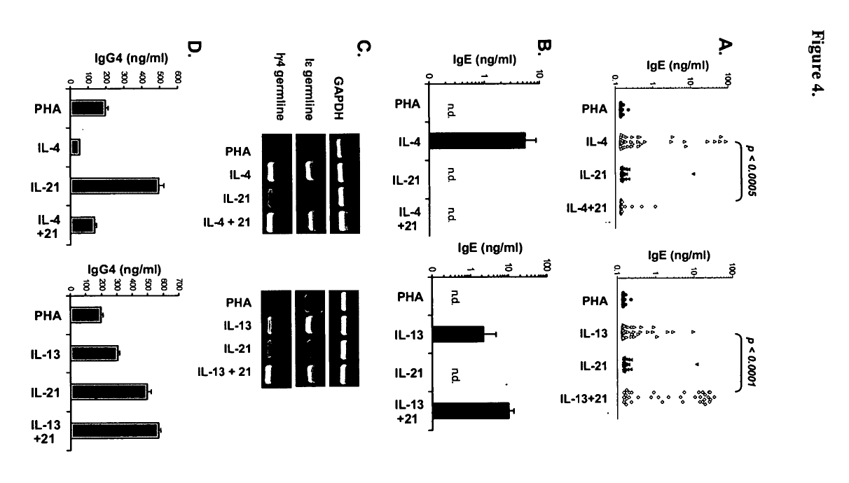

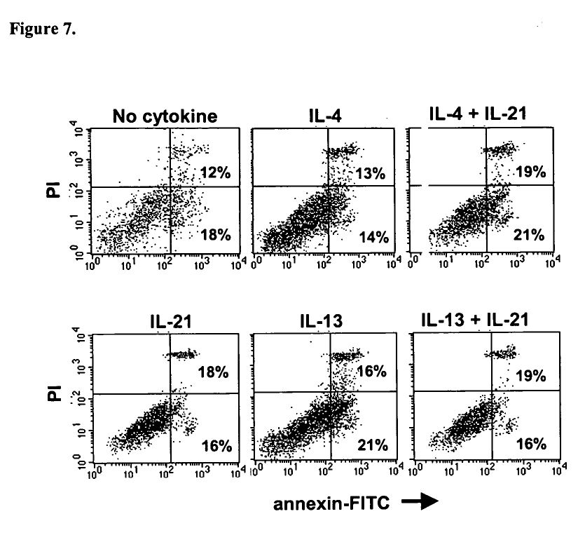

A variety of assays are available to evaluate a candidate agent, e.g., for use as an IL-21 pathway agonist or an IL-21 pathway antagonist. Exemplary activity assays for IL-21 polypeptides and IL-21 receptors proteins are described, e.g., in Kasaian et al. (2002) Immunity 16:1-20. These assays can be used to evaluate functionality of an IL-21 polypeptide or other agent. For example, an IL-21 polypeptide may have activity (e.g., at least 25, 50, 75, 80 or 95% specific activity of wild-type) in one or more of the following assays from Kasaian et al. (2002), supra: the T cell proliferation assay (e.g., as in FIG. 7A of the aforementioned reference), IFN-γ production (e.g., as in FIG. 7C of the aforementioned reference), and the NK cytotoxicity assay (e.g., as in FIG. 4 of the aforementioned reference, in the presence of IL-15).

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. U.S.A. 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. U.S.A. 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowman et al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J. E.e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad. Sci. U.S.A. 88:7548-7551, 1991.

Assays for evaluating activation of STAT are described, e.g., in Gilmour et al. (1996) Proc. Natl. Acad. Sci. USA 92:10772-10776. For example, evaluated cells (e.g., cells treated with an agonist or a candidate agonist) can be lysed and tyrosine phosphorylated proteins can be immunoprecipitated with an anti-phosphotyrosine antibody. Then precipitated materials can then be evaluated using antibodies specific for a signaling pathway component, e.g., an antibody to the STAT protein, e.g., STAT5.

Assays for Evaluating Cytokine Levels

Any standard assay can be used to evaluate cytokine levels in a sample or a subject, e.g., to evaluate an IL-21 parameter. For example, the sample can be obtained from a subject or can include culture cells. Exemplary samples can be obtained or derived from one or more cells, tissue, or bodily fluids such as blood, urine, lymphatic fluid, cerebrospinal fluid, or amniotic fluid, cultured cells (e.g., tissue culture cells), buccal swabs, mouthwash, stool, tissues slices, and biopsy materials (e.g., biopsy aspiration).

Methods for evaluating cytokine levels include evaluating nucleic acids to detect mRNA or cDNA encoding a cytokine of interest (e.g., IL-21) or evaluating proteins to detect the cytokine itself. Nucleic acids can be evaluated, e.g., using RT-PCR (e.g., quantitative PCR) or nucleic acid microarrays. Proteins can be evaluated, e.g., using mass spectroscopy or an immunoassay.

ELISAs provide one convenient form of immunoassay. For example, Biosource International, Camarillo Calif. provides assay reagents that can be used to detect IL-21, IL-10, and to IL-12. Similarly, R&D Systems provides reagents to detect IFN-γ with a sensitivity<8 pg/ml or TGF-beta1 with a sensitivity of <7 pg/ml. SEARCHLIGHT™ Proteome Array System (Pierce, Boston Technology Center) provides comprehensive reagents for evaluating multiple cytokines at once.

These methods can be used to evaluate a subject, e.g., before, during, or after administration of an IL-21 pathway modulator (e.g., agonist or antagonist). For example, to determine if such agonist causes a statistically significant change in the levels of a cytokine, e.g., IL-21, IL-10 or IFNγ or to determine if it causes an acceptable changes, e.g., to a level in a range of normal of a cytokine, e.g., IL-21, IL-10 or IFNγ. Information from the evaluating can be used to modulate the dosage of the agonist.

Similarly, methods for evaluating IgG and IgE levels are available. For example, Alpha Diagnostic International, Inc. (San Antonio, Tex.) provides an ELISA kit for evaluating human IgE, as does Bethyl Laboratories, Inc. In one embodiment, if IgE levels are not decreased to levels within the range of a normal subject, administration of the IL-21 agonist can be increased, e.g., by increasing dosage or frequency, e.g., by a proportional or corresponding amount, or by at least about 1.5, 1.8, or 2 fold.

### EXAMPLE

In human atopic disease, IgE sensitizes the allergic response, while IgG4 is protective. Because IL-4 and IL-13 trigger Ig switch recombination to both IgE and IgG4, additional agents may regulate the balance between these isotypes to influence susceptibility or tolerance to atopy. IL-21 reduces IL-4-driven IgE switch recombination but increases IgG4 secretion by human PBMC. In contrast to its effects in the murine system, IL-21 inhibition of human IgE production was not a direct effect on B cells, and could be overcome by cross-linking B cell CD40 with anti-CD40 antibody. Furthermore, IL-21 did not block IgE produced in response to IL-13. T cells respond to IL-4 but not IL-13, and T cell expansion appears to contribute to the inhibitory effects of IL-21 on IgE production. Neither IFN-γ, IL-10, IL-12, CD40 expression nor apoptosis was responsible for the inhibitory effect.

In contrast to its indirect inhibition of IgE production, IL-21 stimulated secretion of IgG4 from PBMC. We found that IL-21 may influence the production of both human IgE and IgG4, and thus contribute to the regulation of atopic reactions.

Materials and Methods

PBMC isolation and culture. Peripheral blood from healthy human donors was drawn into heparinized VACUTAINER™ tubes (BD, Mountain View, Calif.). Mononuclear cells were isolated by centrifugation over HISTOPAQUE-1077™ (Sigma, St. Louis, Mo.). For culture of whole PBMC, cells were plated at 2×10⁶/ml in 96-well round-bottom plates containing 1×10⁶/ml irradiated (1500 RAD) autologous PBMC as feeders, in RPMI containing 10% heat-inactivated FCS, 50 U/ml penicillin, 50 μg/ml streptomycin, 2 mM L-glutamine. For PHA activation, PBMC were plated with 2 μg/ml PHA-P (Sigma). After 2 days, fresh media was added lacking PHA but containing 25 ng/ml recombinant human IL-4 or 50 ng/ml recombinant human IL-13, +/−20 ng/ml recombinant human IL-21 (R&D Systems Inc., Minneapolis, Minn.). These levels were determined by dose titration for each cytokine. For anti-CD40 monoclonal antibody activation, PBMC were plated with 1 μg/ml anti-human CD40 (BD Pharmingen) in the presence of cytokines. For both PHA- and anti-CD40-activated cultures, media containing fresh cytokines was added every 4 days. On day 14-21 of PHA cultures, or day 6-12 of anti-CD40 monoclonal antibody cultures, media was harvested for determination of antibody levels. These time points reflect the more rapid time course for IgE production under conditions of anti-CD40 treatment compared to PHA stimulation. Cells were isolated early (day 3-5) or later (day 10-14) in the course of culture for RNA isolation.

B cell enrichment. PBMC isolated as described above were incubated with B cell enrichment cocktail (ROSETTESEP™, StemCell Technologies, Vancouver, British Columbia, Canada), and B cells isolated according to the manufacturer's instructions. The resulting population was >88% CD20+ B cells. B cells were plated at 2×10⁵/ml in media containing 1×10⁶/ml irradiated (1500 RAD) autologous PBMC as feeders, and treated with anti-CD40 monoclonal antibody or cytokines as described above. On day 6-12 of culture, media was harvested for determination of antibody levels.

ELISA for human Ig isotypes. ELISA plates (EIA/RIA plates; Corning Costar, Acton, Mass.) were coated with 1 μg/ml goat anti-human IgE (KPL Inc., Gaithersburg Md.) or 3 μg/ml mouse anti-human IgG4 (Southern Biotechnology Associates, Birmingham, Ala.) in 0.1M sodium carbonate, 0.1M sodium bicarbonate buffer, pH 9.6 overnight at 4° C. Plates were blocked for 1 hour with 0.5% gelatin and 1% polyvinylpyrrolidine (Sigma, St. Louis, Mo.) in PBS. Plates were washed with PBS containing 0.05% Tween-20 (PBS-Tween), then incubated with serum or human IgE (Biodesign Int, Kennebunk, Me.) or IgG4 (Sigma) isotype standards for 4 hours at room temperature. After washing with PBS-Tween, plates were incubated for 2 hours at room temperature with biotinylated antibody directed against human IgE (KPL) or IgG4 (Southern Biotechnology Associates). Plates were washed and incubated with HRP-labeled streptavidin (Southern Biotechnology Associates) for 1 hour at room temp. Plates were washed and incubated with the peroxidase substrate Sure Blue (KPL). The reaction was stopped by adding 0.1N HCL, and absorbance at 450 nm was read in a SPECTRAMAX™ plate reader (Molecular Devices Corp., Sunnyvale, Calif.). In order to demonstrate isotype specificity, purified human IgM, IgG isotypes, or IgA (BD Biosciences Pharmingen, San Diego, Calif.) were run in the IgE and IgG4 ELISAs and produced no signal. The limit of sensitivity of the IgE ELISA was 0.3 ng/ml. The limit of sensitivity of the IgG4 ELISA was 4 ng/ml.

Cytokine analysis. Cytokine levels in culture supernatants were determined using assay kits for IL-10 (Biosource International, Camarillo, Calif.; sensitivity<0.2 pg/ml), IL-12 (Biosource International; sensitivity<2 pg/ml), IFN-γ (R&D Systems; sensitivity<8 pg/ml) or TGF-β1 (R&D Systems, sensitivity<7 pg/ml).

Proliferation Assays. Enriched human B cells were cultured in RPMI containing 10% FBS, 50 U/ml penicillin, 50 μg/ml streptomycin, 2 mM L-glutamine at 2×10⁵/well in 96-well round-bottom plates. Anti-CD40 monoclonal antibody and cytokines were added as described above. On day 3, cultures were pulsed with 0.5 μCi/well 3H-thymidine (PerkinElmer NEN, Boston, Mass.), and harvested 5 hours later onto glass fiber filter mats. 3H-thymidine incorporation was determined by liquid scintillation counting.

Apoptosis assay. Apoptotis was measured by flow cytometry using a Annexin V-FITC Apoptosis Detection Kit (Calbiochem, La Jolla, Calif.). PBMC were cultured as described above, and apoptosis measured at 24 and 48 hours following addition of cytokines. Cells were incubated with annexin V-FITC and APC-conjugated anti-human CD19 (BD Pharmingen) for 15 minutes at room temperature and washed. Propidium iodide was added and fluorescence was analyzed using a BD FACSCalibur cytometer and CellQuest software (BD Biosciences).

RNA isolation. On day 5 of PBMC or B cell cultures, cells were pooled from microtiter wells, washed with PBS, lysed with RLT buffer (Qiagen Inc., Valencia, Calif.), and prepared with QIASHREDDER™. RNA was prepared using the RNA MINI™ Kit (Qiagen) according to manufacturer's instructions.

Reverse-transcription and PCR analysis of sterile transcripts. mRNA prepared as described above was transcribed to cDNA using the Promega Reverse Transcription kit (Promega Corp., Madison, Wis.). PCR was performed using the Clontech ADVANTAGE™ PCR kit (BD Biosciences Clontech, Palo Alto, Calif.) and the following primer sequences and conditions. GAPDH was amplified in 25 cycles of 1 minute each at 94° C., 65° C., and 72° C. using the primers. Iε germline transcript was amplified in 38 cycles of 1 minute each at 94° C., 65° C., and 74° C. using primers (42). Iγ4 germline transcript was amplified in 38 cycles of 1 minute each at 94° C., 65° C., and 76° C. using primers (43). Mature IgE transcripts were amplified in 38 cycles of 1 minute each at 94° C., 69° C., and 74° C. using a JH consensus forward primer: 5′ (44) combined with the Iε reverse primer. Primers were prepared by Eurogentec (San Diego, Calif.). Amplified products were run on 1.2% agarose gels containing ethidium bromide.

Real time RT-PCR. Total RNA was isolated from cells using the RNEASY™ Mini kit (Qiagen, Valencia, Calif.). Oligonucleotides were designed to human GAPDH, IL-12p35, IL-10 and IL-12Rβ2 using PRIMER EXPRESS™ software (Applied Biosystems Division of Perkin Elmer Corp., Foster City, Calif.) and synthesized by Eurogentec. Probes were labeled on the 5′ end with the reporter dye, 6-carboxyfluorescein (FAM) and on the 3′ end with the quencher dye 6-carboxy-tetramethylrhodamine (TAMRA). Reactions were set up using a reverse transcriptase q-PCR MASTERMIX™ (Eurogenetec) and 50 ng of template RNA per reaction. Samples were run in duplicate on the PRISM 7000™ Sequence Detection System (Applied Biosystems) using the following RT-PCR program: (1) 30′ cycle at 48° C., (50) 10′ cycles at 95° C., (1) 15″ cycle at 95° C. and (1) 1′ cycle at 60° C. Data was analyzed using PRISM 7000™ software. Each result was fit to a standard curve generated from a positive control source of RNA and expression values were normalized to GAPDH.

Statistical analysis. All observations were reproduced in 2-6 separate experiments. Data between treatment groups were compared using student's t-test. For analysis cytokine effects on IgE production in microcultures, the IgE level in each microwell with a given treatment was taken as a separate determination, with n=24-36 per treatment. For analysis of cytokine effects on IgE or cytokine production in bulk cultures, replicate cultures were established per treatment. p values of <0.05 were considered significant.

Results:

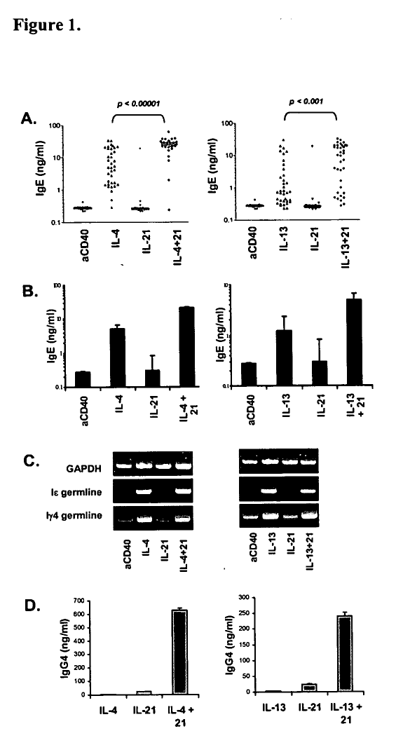

IL-21 enhances IL-4- and IL-13-driven IgE synthesis in human B cells. IgE switch recombination can be triggered by exposure of B cells to a CD40 cross-linking agent in the presence of IL-4 or IL-13. In order to investigate the effects of IL-21 on this process, B cells were enriched from human PBMC to >88% purity, and stimulated with anti-CD40 mAb in the presence of IL-4 or IL-13. CD3+ cells were undetectable. Individual cultures were established in 24-36 microtiter wells per treatment. In the absence of IL-4 or IL-13, none of the wells contained IgE, consistent with a lack of detectable IgE-producing cells. When IL-4 or IL-13 was added, most of the microcultures contained IgE-producing cells (FIG. 1A), with detectable IgE in the supernatant (FIG. 1B). Although limiting dilution analysis was not performed to calculate the exact frequency, an increase in the number of IgE-positive microcultures was taken to indicate an increased frequency of IgE-producing B cells. Addition of IL-21 to IL-4 or IL-13 consistently increased IgE production over levels seen with IL-4 or IL-13 alone (FIG. 1A, B). The percentage of IgE-producing wells was virtually 100% with IL-4 or IL-4+IL-21, and increased from 61% with IL-13 alone to 78% with IL-13+IL-21. IL-4 and IL-13 also induced production of the Iε germline transcript (FIG. 1C), which is associated with de novo Ig switch recombination to the Cε locus.

IL-4 and IL-13 also induced generation of the Iγ4 germline transcript (FIG. 1C), but in our culture system, IL-4 or IL-13 alone was not sufficient to support IgG4 production and release from the cells (FIG. 1D). In contrast, IL-21 alone generated only background levels of Iγ4 germline transcript (FIG. 1C), but did stimulate low levels of IgG4 release into the supernatant of anti-CD40 mAb-treated B cells. Addition of IL-21 to IL-4 or IL-13 strongly enhanced IgG4 production over levels seen with IL-4 or IL-13 alone (FIG. 1D). In fact, very little IgG4 was released from the cells unless IL-21 was added to the cultures.

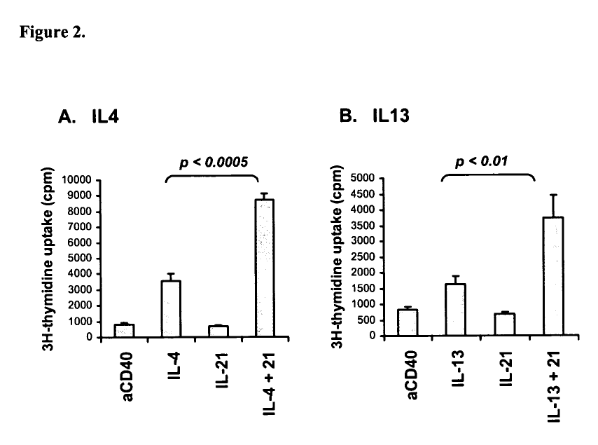

IL-21 stimulates proliferation of human B cells that have been treated with anti-CD40 mAb (22), and the proportion of cells undergoing isotype switch recombination increases with cell division (34). In order to determine if increased B cell proliferation could help to account for the enhanced levels of IgE and IgG4 seen in the presence of IL-21, we evaluated 3H-thymidine incorporation by purified B cells under the culture conditions used above. Results show that IL-21 enhanced B cell proliferation above levels seen with IL-4 or IL-13 alone (FIG. 2).

IL-21 enhances IgE synthesis in unfractionated PBMC stimulated with anti-CD40 mAb and IL-4 or IL-13. In addition to its reported effects on B cells, IL-21 has potent effects on human T cells. It induces T cell proliferation (22, 23, 35), and potentiates cytokine production in the presence of TCR cross-linking agents and appropriate costimulation (23, 36). Therefore, it was of interest to investigate IL-21 effects on IgE production under conditions in which T cells were also present and could respond to the cytokine.

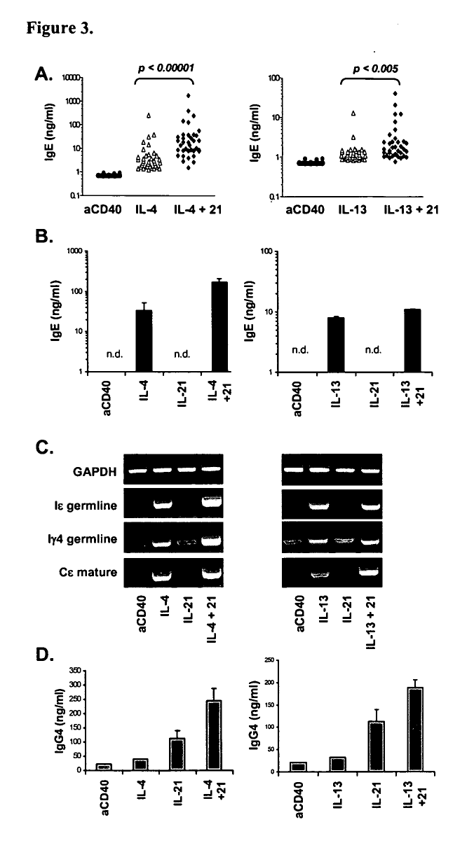

Unfractionated PBMC were treated with anti-CD40 mAb in combination with IL-4 or IL-13 to drive IgE production. IgE was measured in the supernatants 7 to 14 days later. In combination with IL-4 or IL-13, IL-21 produced a modest increase in levels of IgE and IgG4 protein (FIG. 3A, B, D). The percentage of IgE-producing wells increased from 86% with IL-4 alone to 100% with IL-4+IL-21, and increased from 19% with IL-13 alone to 56% with IL-13+IL-21. Consistent with this, IL-4 or IL-13 induced Iε germline transcript, J-Cε mature transcript, and Iγ4 germline transcript all were maintained in the presence of IL-21 (FIG. 3C).

IL-21 blocks IgE synthesis in unfractionated PBMC stimulated with PHA and IL-4 Activated T cells are the only known source of IL-21. In the next series of experiments, effects of IL-21 were investigated under conditions in which Ig class switch recombination was dependent on T cell activation. Unfractionated PBMC were treated with the T cell mitogen, PHA, to induce CD40L expression (51). Upon addition of IL-4 or IL-13, IgE was released into the supernatant within 14-21 days. In this T cell-dependent system, IL-4-driven IgE production was blocked by IL-21, which greatly reduced the levels of IgE released into the supernatant (FIG. 4A,B). The percentage of IgE-producing wells decreased from 47% with IL-4 alone to 6% with IL-4+IL-21. Interestingly, this effect was not seen when IgE synthesis was initiated with IL-13. Cells treated with IL-13+IL-21 produced more IgE than those treated with IL-13 alone (FIG. 4A,B), as had been seen with purified B cells (FIG. 1A). The percentage of IgE-producing wells increased from 31% with IL-13 alone to 68% with IL-13+IL-21. Taking into account that T cells respond to IL-4 but not to IL-13, these observations point to a T cell-dependent mechanism for the inhibitory activity of IL-21 on IL-4-driven IgE production in this system.

To further investigate this inhibitory activity, Is germline transcription was examined. With PHA stimulation, Is germline transcript was detectable early after addition of IL-4 or IL-13 (days 3-5 of culture). Although IL-21 blocked IgE production in IL-4-treated cultures, it did not prevent this initial induction of Iε germline transcript by either IL-4 or IL-13 (FIG. 4C).

IL-21 increases IgG4 production in unfractionated PBMC stimulated with PHA. In PHA-stimulated PBMC cultures, treatment with IL-4 or IL-13 induced high levels of Iγ4 germline transcript. PBMC treated with IL-21 or with no added cytokine also showed detectable transcript (FIG. 4C). By day 14-15, much higher levels of IgG4 were found in cultures that had been treated with IL-21 than in those treated with IL-4 or IL-13 alone (FIG. 4D). Addition of IL-4 was inhibitory for IL-21-induced IgG4 production, whereas addition of IL-13 was not (FIG. 4D).

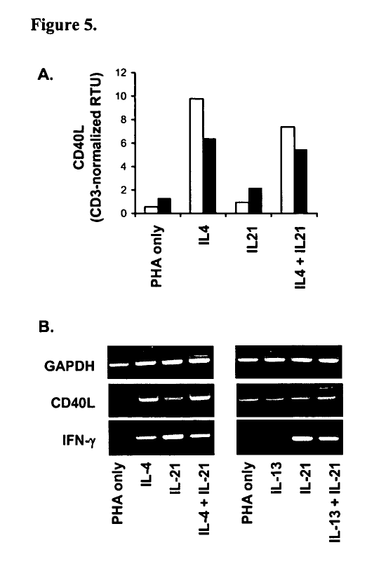

CD40L expression is maintained in the presence of IL-21. An inhibitory effect of IL-21 on IgE and IgG4 production was seen when PHA was used to induce co-stimulatory signals for IL-4-driven IgE production (FIG. 4). In contrast, when anti-CD40 was used to directly cross-link CD40 in PBMC cultures, IL-21 did not block IgE production in response to IL-4 (FIG. 3). Thus, we considered the possibility that IL-21 reduced CD40L expression by PBMC stimulated with PHA and IL-4. CD40L mRNA is labile, and expression is thought to be transcriptionally regulated (37, 38). Using real time PCR, we examined CD40L transcript levels in PHA-stimulated PBMC cultures early after addition of cytokine (day 4), or at a later time point (day 14), at which IgE was measurable in the cell supernatants. At both time points, cells treated with IL-4 or IL-4+IL-21 showed strong CD40L mRNA expression, while transcript levels with IL-21 alone were not elevated over those seen with PHA (FIG. 5A). These findings were supported by PCR amplification using primers spanning the entire CD40L coding region (FIG. 5B). This result clearly demonstrates that the presence of IL-21 does not block CD40L transcription, indicating that CD40L expression is not reduced under conditions in which IgE production is inhibited.

IL-21 induces expression of IFN-γ. Several experiments were done to address whether treatment of PHA-stimulated, IL-4-activated PBMC with IL-21 resulted in generation of cytokines that block IgE production. The ability of IFN-γ to antagonize IgE synthesis has been well-characterized (10, 13, 14, 39), and IL-21 is known to stimulate IFN-γ gene transcription in human T and NK cells (36, 40). Therefore, the expression of IFN-γ transcript was examined in PHA-stimulated PBMC treated with IL-4, IL-113, or IL-21. Early in the cultures, when Iε germline transcript was detectable, IFN-γ gene expression was seen under all treatment conditions. By day 14 of culture, when IgE could be assayed from the supernatant, IFN-γ gene expression was seen only in cultures treated with IL-4 or those treated with IL-21 (FIG. 5). Thus, IFN-γ transcripts were found both in cultures treated with IL-4+IL-21, in which IgE production was reduced, and in those treated with IL-13+IL-21, in which IgE production was maintained.

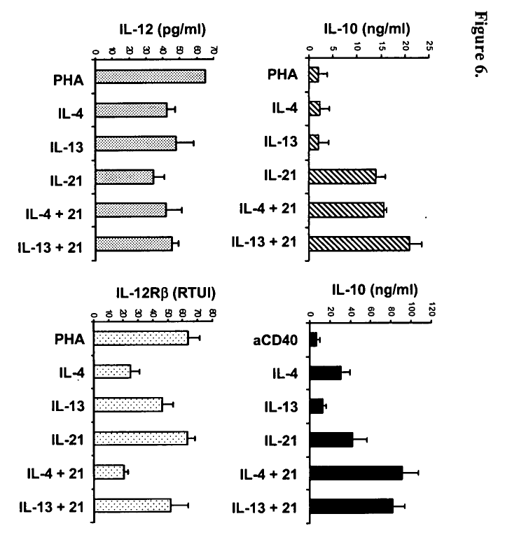

IL-21 induces IL-10 production by PBMC but does not affect production of IL-12 or expression of IL-12Rβ. IL-10 is a multi-potent cytokine that has been reported to stimulate (41) or inhibit (21) B cell IgE synthesis, depending on the presence of other cytokines or co-stimulatory signals. We asked whether IL-10 was produced in IL-21-treated PBMC cultures and could help to explain the inhibition of IgE production that was seen in the presence of IL-4. IL-21 was found to boost IL-10 production by PBMC, both in PHA-stimulated cultures (FIG. 6A), where IgE production was inhibited (FIG. 4), and in anti-CD40 mAb-stimulated cultures (FIG. 6B), where IgE production was not inhibited (FIG. 3). Furthermore, comparable IL-10 levels were seen in the presence of IL-4 or IL-13 (FIG. 6A,B). Real-time PCR analysis confirmed that IL-21 increased IL-10 production, but the increase was seen whether or not IgE was released. To address the role of IL-10 more directly, neutralizing antibody to IL-10 was added to the PHA-stimulated cultures, and did not overcome the inhibitory effect of IL-21 on IgE production.

Several other cytokines have been reported to block IgE production, including IL-12 (19), and TGF-beta (10). Both IL-12 (FIG. 6C) and TGF-beta could be detected in PBMC cultures, but levels were similar in cells treated with IL-4 or IL-13, in the presence or absence of IL-21. IL-21 also had no effect on IL-12Rβ gene expression induced by PHA, IL-4, or IL-13 (FIG. 6D). Thus, neither IFN-γ, IL-10, IL-12, nor TGF-beta could satisfactorily account for the inhibitory effect of IL-21 on IL-4-driven IgE production.

IL-21 does not drive B cell apoptosis in PHA-stimulated PBMC cultures. IL-21 has been shown to induce apoptosis of primary murine B cells (25). Thus, it is possible that B cells of PHA-stimulated PBMC cultures treated with IL-21 were driven to apoptosis, accounting for the decrease in IgE production. In order to address this issue, PHA-stimulated PBMC were stained with anti-CD19 to identify B cells, and assayed for binding of PI and FITC-annexin by flow cytometry. Late apoptotic cells (PI⁺/FITC-annexin⁺) could be distinguished from early apoptotic (PI^{neg}/FITC-annexin⁺) or viable (PI^{neg}/FITC-annexin^{neg}) B cells. Results show that addition of IL-21 resulted in a minor increase in the percentage of apoptotic CD19+ cells in IL-4-treated cultures, but that the level of apoptosis was not different than that seen in cultures treated with IL-13 or IL-13+IL-21 (FIG. 7). Thus, induction of B cell apoptosis does not account for the inhibitory effect of IL-21 on IgE production.

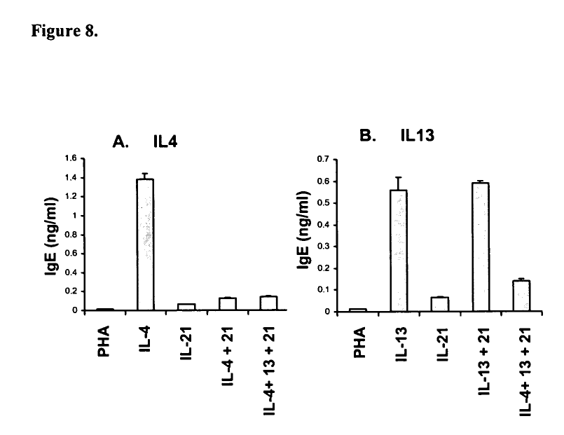

Adding back IL-13 does not restore IgE production in PBMC treated with IL-4 and IL-21. Because IL-21 inhibited IgE production from PHA-stimulated PBMC in response to IL-4 but not IL-13 (FIG. 4), we asked whether the presence of all three cytokines would have a net activating or inhibitory effect. Results showed that the combination of IL-4 and IL-13 was inhibitory for IgE production. Thus, IL-13 did not rescue IgE production from PHA-stimulated PBMC treated with IL-4 and IL-21 (FIG. 8A), whereas addition of IL-4 reduced the IgE production that was normally seen in PHA-stimulated PBMC treated with IL-13 and IL-21 (FIG. 8B).

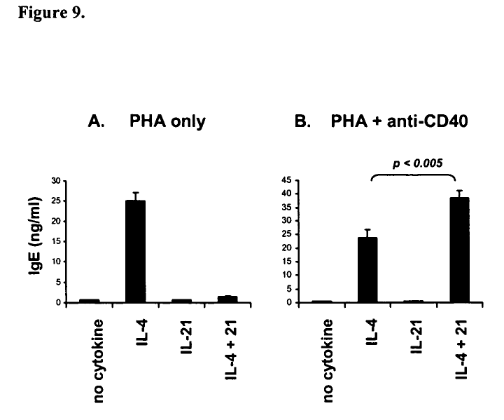

CD40 ligation overcomes the inhibitory effect of IL-21 on IgE production. We have observed that in human PBMC stimulated with anti-CD40 and IL-4, the addition of IL-21 boosts IgE production (FIG. 3). In contrast, in PBMC stimulated by PHA and IL-4, the addition of IL-21 blocks IgE production (FIG. 4). In order to help reconcile these observations, PHA-activated PBMC were treated with anti-CD40 in combination with IL-4 in the presence or absence IL-21. Under these conditions, IL-21 did not inhibit IgE production, but rather boosted levels of IgE above those seen with IL-4 alone (FIG. 9). Thus, anti-CD40 was able to overcome the inhibitory effect of IL-21 on IgE production by mitogen-activated PBMC.

IL-21 does not reduce IgE production by PHA-stimulated irradiated PBMC. In these studies, PHA-stimulated T cell expansion was greatly potentiated by the combination of IL-4+IL-21. Because T cells can respond to IL-4, it is possible that IL-4 becomes depleted from these cultures. According to this scenario, initial Iε transcript can be seen on days 3-5 (FIG. 4C), but once T cell numbers become too high, the IL-4 levels cannot sustain B cell IgE or IgG4 production. Because T cells do not interact with IL-13, this cytokine would not be depleted, and B cell IgE production could be sustained in PHA and IL-13-treated cultures.

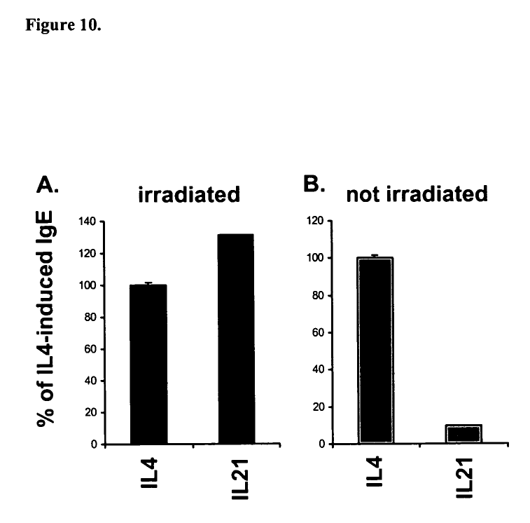

In order to test the hypothesis that T cell expansion contributes to the reduced IgE production in PHA-stimulated cultures treated with IL-4 and IL-21, PBMC were irradiated following PHA stimulation. Purified B cells were added back to comprise 20% of the culture, to approximate the B cell frequency of normal PBMC. The cells were treated with cytokines as above, and IgE production was examined on day 13. With T cell expansion prevented by irradiation, the addition of IL-21 did not reduce IL-4 mediated IgE production (FIG. 10A). In non-irradiated cultures set up in parallel, however, IL-21 did result in decreased IgE production (FIG. 10B), in agreement with results shown in FIG. 4A,B. These observations suggest that the apparent decrease in IgE production resulting from addition of IL-21 to IL-4-treated; PHA-stimulated PBMC was secondary to lymphocyte expansion and was not a direct effect on the B cells.

Discussion

IgE switch recombination in vitro requires two distinct signals: (i) the cytokines IL-4 or IL-13 to drive generation of the Iε germline transcript; and (ii) engagement of the B cell surface CD40 antigen to promote deletional switch recombination (42). Cytokines provide important regulation of this process. IL-21 has been shown to inhibit IgE production in murine systems (26, 27), but its effects on human IgE production have not been explored in detail. We have examined the effects of IL-21 on human IgE production under three different models of activation and found that, depending on the conditions, IL-21 can be stimulatory or inhibitory.

IL-21 is a pleiotropic cytokine produced by activated T cells, that has effects on many immune cell types (22, 23). Under appropriate conditions, it induces B cell proliferation (22) or B cell apoptosis (25). In murine systems, IL-21 blocks IgE production both in response to IL-4 and mitogen stimulation in vitro, and specific immunization in vivo (26, 27). Accordingly, IL-21R-deficient mice have increased resting levels of serum IgE compared to wild-type mice (23), and produce higher levels of IgE upon immunization or infection (26). In isolated murine B cells, IL-21 directly antagonizes IL-4 and LPS-induced Iε switch recombination (27).

We now report that IL-21 enhances IL-4- or IL-13-mediated IgE production by isolated human B cells. IL-21 potentiated IgE synthesis not only by purified B cells but also by IL-4- or IL-13-treated PBMC in which B cell activation was achieved with anti-CD40 mAb. Resting human peripheral blood B cells express IL-21 receptor, and IL-21 can potentiate anti-CD40-induced B cell proliferation (22). We observed increased 3H-thymidine incorporation by IL-4 or IL-13 treated B cells in the presence of IL-21. Thus, the enhancement of IgE production seen in the presence of IL-21 may be a consequence, at least in part, of IL-21-mediated B cell expansion.

In contrast, an inhibitory effect of IL-21 was observed when PHA-activated T cells were used as the source of costimulatory signals for IgE production. Under these conditions, IL-21 blocked IgE synthesis driven by IL-4 but not IL-13. Although not conclusive, these observations point to a T cell-dependent mechanism, as PHA is a T cell mitogen and T cells respond to IL-4 but not IL-13. Because anti-CD40 antibody could overcome the inhibition, CD40L function or expression is implicated. Moreover, we observed Iε germline transcript in the absence of IgE synthesis, which is characteristic of defects in CD40L expression (43, 44) or CD40 signalling (45). Nevertheless, CD40L transcripts, which are labile and limiting for protein expression (37, 38) were not decreased by IL-21. Thus, we speculate that IL-21 may elicit additional cell surface signals that block T cell-B cell interaction in this system, or reduce the strength of the CD40L signal.

Several cytokines have been described to antagonize IgE production, including TGF-beta (10, 46, 47), IFN-γ (10, 13, 14, 46), IL-10 (21, 48), and IL-12 (18). We compared levels of these cytokines in cultures in which IgE was produced or inhibited. TGF-beta was detected in all cultures, but showed no association with IgE levels. IFN-γ transcription was elicited by IL-21 in PHA-stimulated PBMC cultures, in agreement with previous reports (36, 40), but was not associated with loss of IgE synthesis. It was maintained with either IL-4+IL-21 treatment, in which IgE production was blocked, or IL-13+IL-21 treatment, in which there was no inhibition.

IL-10 blocks IgE production in a monocyte-dependent manner, such that it has no inhibitory activity on purified B cells (49), similar to the current findings with IL-21. Although IL-10 was found in PBMC cultures, it was not associated with inhibition of IgE production. Equivalent levels were seen in cultures stimulated with anti-CD40 mAb or PHA, although IgE was only inhibited with PHA. In PHA-treated PBMC, comparable levels of IL-10 were produced with IL-4+IL-21, which was inhibitory for IgE production, and with IL-13+IL-21, which was stimulatory. Neutralizing antibody to IL-10 did not reverse the inhibitory effect of IL-21. These observations indicate that IL-10 is not responsible for the effects of IL-21 seen in this system.

IL-12 has also been reported to reduce IL-4-driven IgE production by unfractionated PBMC, but not by purified B cells (18), similar to the current observations with IL-21. Furthermore, IL-21 may influence lymphocyte responses to IL-12. IL-21 up-regulates transcription of IL-12Rβ2 in a human NK cell line and in primary human T cells (40), greatly enhances IL-12-mediated IFN-γ secretion by mouse NK cells (23), and promotes IL-12-mediated STAT4 binding to the IFN-γ-activated sequence of the IL2Rα gene (40).

IL-21 drives apoptosis of murine B cells, even those that have been stimulated with LPS (25, 50). IL-4 cannot rescue IL-21-treated B cells from apoptosis, but pre-activation with anti-CD40 mAb is protective (25). Thus, unfractionated PBMC stimulated with IL-4 and PHA may have reduced IgE production because the B cells had undergone apoptosis, whereas those stimulated with anti-CD40 mAb were protected. We found apoptosis of B cells in PBMC cultures, which was marginally increased with IL-21, but addition of IL-4 produced no more apoptosis than IL-21 alone or IL-13+IL-21. Thus, conditions leading to reduced IgE production were not associated with enhanced B cell apoptosis.

Mice lacking either IL-4 or IL-13 do not generate wild-type levels of IgE (51, 52), suggesting that one cytokine alone cannot fully compensate for loss of the other. Indeed, IL-13 may be the major driver for atopic responses, as selective neutralization (53) or deletion (54) of IL-13 protected mice from development of asthma pathology despite the presence of IL-4. Recently, Hajoui et al. (55) have shown that IL-13 production by B cells themselves is required for generation of IgE in response to IL-4, and propose that B cell production of IL-13 is necessary for IL-4-induced IgE synthesis. We observed IL-13 production in PHA-stimulated PBMC treated with IL-4, which was reduced by half with added IL-21. Furthermore, our findings that IL-21 antagonized IL-4-induced IgE generation in PHA-stimulated PBMC, while the IL-13 response remained robust, could signal a role for IL-21 in regulation of this autocrine pathway.

Whereas IgE directed against allergens is necessary and sufficient for development of atopic disease (56), IgG4 has long been thought to be protective (4, 5, 6, 7). We found that treatment of mitogen-activated PBMC with IL-21 alone stimulated IgG4 release. No IgE was produced under these conditions, suggesting that IL-21 may be capable of shifting the balance between IgG4 and IgE under appropriate circumstances. In contrast, although IL-4 or IL-13 generated high levels of I□4 germline transcript, we and others (57) found that these cytokines alone did not result in detectable IgG4 protein release from human peripheral B cells. Ig gene rearrangement and antibody secretion are differentially regulated events (58). IL-4 induces IgG4 switch recombination in naïve B cells, but may repress secretion of mature protein in those cells that have already switched to IgG4 (59). The opposite was seen with IL-21 alone, which did not induce Iγ4 germline transcript above unstimulated levels, but strongly enhanced secretion of IgG4 protein. IL-21 induces secretion of all human IgG isotypes, while promoting de novo switch recombination specifically only to IgG1 and IgG3 (57). Release of protein without de novo transcription suggests that IL-21 promoted the activation or expansion of B cell clones that had been committed in vivo to generation of IgG4, a process previously shown to account for the IL-4/IL-13-independent generation of IgG4 in vitro (60).

Taken together, these studies show that IL-21 stimulates or inhibits IgE and IgG4 production by human B cells depending on activation conditions. IL-21 has similarly contradictory responses in other systems. Under appropriate conditions, it can induce NK cell activation and/or apoptosis, stimulate or limit T cell expansion, and induce or inhibit T cell IFNγ production (61). In the murine system, IL-21 triggers apoptosis of B cells treated with LPS, but co-stimulates proliferation of B cells treated with anti-CD40 or anti-IgM (25, 50, 62). It has been proposed that IL-21 acts as a checkpoint for productive immune responses, driving activation and proliferation under permissive conditions, while promoting apoptosis of lymphocytes activated inappropriately or in an unfavorable environment (50, 61, 62). In the context of the current study, IL-21 appears to exert a regulatory influence over human IgE production, either boosting levels or ensuring against over-production of this critical effector molecule.

### REFERENCES

1. T. Kawakami, S. J. Galli, Regulation of mast-cell and basophil function and survival by IgE, Nat. Rev. Immunol. 2 (2002) 773-786.
2. C. Prussin, D. D. Metcalfe, IgE, mast cells, basophils, and eosinophils, J. Allergy Clin. Immunol. 111(2 Suppl) (2003) S486-S494.
3. W. Busse, W. Neaville, Anti-immunoglobulin E for the treatment of allergic disease, Curr. Opin. Allergy Clin. Immunol. 1 (2001) 105-108.
4. D. Stern, W. Eder, G. Tebow, I. C. Lohman, C. Braun-Fahrlander, J. Riedler, D. Nowak, E. Soprana, E. Von Mutius and the ALEX Study Group, M. Halonen, D. Vercelli, Rethinking Th2 antibody responses and allergic sensitization. Novartis Found. Symp. 257 (2004) 25-37.
5. T. Platts-Mills, J. Vaughan, S. Squillace, J. Woodfolk, R. Sporik, Sensitisation, asthma, and a modified Th2 response in children exposed to cat allergen: a population-based cross-sectional study, Lancet 357 (2001) 752-756.
6. A. Custovic, C. S. Murray, The effect of allergen exposure in early childhood on the development of atopy, Curr. Allergy Asthma Rep. 2 (2002) 417-423.
7. T. A. Platts-Mills, J. W. Vaughan, K. Blumenthal, S. Pollart Squillace, R. B. Sporik, Serum IgG and IgG4 antibodies to Fel d 1 among children exposed to 20 microg Fel d 1 at home: relevance of a nonallergic modified Th2 response, Int. Arch. Allergy Immunol. 124 (2001) 126-129.
8. R. S. Geha, H. H. Jabara, S. R. Brodeur, The regulation of immunoglobulin E class-switch recombination, Nat. Rev. Immunol. 3 (2003) 721-732.
9. S. Monticelli, D. Vercelli, Molecular regulation of class switch recombination to IgE through epsilon germline transcription, Allergy 56 (2001) 270-278.
10. J. F. Gauchat, G. Aversa, H. Gascan, J. E. de Vries, Modulation of IL-4 induced germline epsilon RNA synthesis in human B cells by tumor necrosis factor-alpha, anti-CD40 monoclonal antibodies or transforming growth factor-beta correlates with levels of IgE production, Int. Immunol. 4 (1992) 397-406.
11. R. J. Armitage, B. M. Macduff, M. K. Spriggs, W. C. Fanslow, Human B cell proliferation and Ig secretion induced by recombinant CD40 ligand are modulated by soluble cytokines, J. Immunol. 150 (1993) 3671-3680.
12. J. Punnonen, J. E. de Vries, IL-13 induces proliferation, Ig isotype switching, and Ig synthesis by immature human fetal B cells, J. Immunol. 152 (1994) 1094-1102.
13. J. Punnonen, K. Punnonen, C. T. Jansen, K. Kalimo, Interferon (IFN)-alpha, IFN-gamma, interleukin (IL)-2, and arachidonic acid metabolites modulate IL-4-induced IgE synthesis similarly in healthy persons and in atopic dermatitis patients, Allergy 48 (1993) 189-195.
14. J. Pene, F. Rousset, F. Briere, I. Chretien, J. Y. Bonnefoy, H. Spits, T. Yokota, N. Arai, K. Arai, J. Banchereau, J de Vries, IgE production by normal human lymphocytes is induced by interleukin 4 and suppressed by interferons gamma and alpha and prostaglandin E2, Proc. Natl. Acad. Sci. USA 85 (1988) 6880-6884.
15. D. Vercelli, H. H. Jabara, K. Arai, T. Yokota, R. S. Geha, Endogenous interleukin 6 plays an obligatory role in interleukin 4-dependent human IgE synthesis, Eur. J. Immunol. 19 (1989) 1419-1424.
16. P. Jeannin, Y. Delneste, S. Lecoanet-Henchoz, D. Gretener, J. Y. Bonnefoy, Interleukin-7 (IL-7) enhances class switching to IgE and IgG4 in the presence of T cells via IL-9 and sCD23, Blood 91 (1998) 1355-1361.
17. B. Dugas, J. C. Renauld, J. Pene, J. Y. Bonnefoy, C. Peti-Frere, P. Braquet, J. Bousquet, J. Van Snick, J. M. Mencia-Huerta, Interleukin-9 potentiates the interleukin-4-induced immunoglobulin (IgG, IgM and IgE) production by normal human B lymphocytes, Eur. J. Immunol. 23 (1993) 1687-1692.
18. B. A. de Boer, B. A., Y. C. Kruize, P. J. Rotmans, M. Yazdanbakhsh, Interleukin-12 suppresses immunoglobulin E production but enhances immunoglobulin G4 production by human peripheral blood mononuclear cells, Infect. Immun. 65 (1997) 1122-1125.
19. M. Kiniwa, M. Gately, U. Gubler, R. Chizzonite, C. Fargeas, G. Delespesse, Recombinant interleukin-12 suppresses the synthesis of immunoglobulin E by interleukin-4 stimulated human lymphocytes, J. Clin. Invest. 90 (1992) 262-266.
20. A. A. Homer, G. F. Widhopf, J. A. Burger, K. Takabayashi, N. Cinman, A. Ronaghy, H. L. Spiegelberg, E. Raz, Immunostimulatory DNA inhibits IL-4-dependent IgE synthesis by human B cells, J. Allergy Clin. Immunol. 108 (2001) 417-423.
21. P. Jeannin, S. Lecoanet, Y. Delneste, J. F. Gauchat, J. Y. Bonnefoy, IgE versus IgG4 production can be differentially regulated by IL-10, J. Immunol. 160 (1998) 3555-3561.
22. J. Parrish-Novak, S. R. Dillon, A. Nelson, A. Hammond, C. Sprecher, J. A. Gross, J. Johnston, K. Madden, W. Xu, J. West, S. Schrader, S. Burkhead, M. Heipel, C. Brandt, J. L. Kuijper, J. Kramer, D. Conklin, S. R. Presnell, J. Berry, F. Shiota, S. Bort, K. Hambly, S. Mudri, C. Clegg, M. Moore, F. J. Grant, C. Lofton-Day, T. Gilbert, F. Rayond, A. Ching, L. Yao, D. Smith, P. Webster, T. Whitmore, M. Maurer, K. Kaushansky, R. D. Holly, D. Foster, Interleukin 21 and its receptor are involved in NK cell expansion and regulation of lymphocyte function, Nature 408 (2000) 57-63.
23. M. T. Kasaian, M. J. Whitters, L. L. Carter, L. D. Lowe, J. M. Jussif, B. Deng, K. A. Johnson, J. S. Witek, M. Senices, R. F. Konz, A. L. Wurster, D. D. Donaldson, M. Collins, D. A. Young, M. J. Grusby, IL-21 limits NK cell responses and promotes antigen-specific T cell activation: a mediator of the transition from innate to adaptive immunity, Immunity 16 (2002) 559-569.
24. A. T. Brenne, T. Baade Ro, A. Waage, A. Sundan, M. Borset, H. Hjorth-Hansen, Interleukin-21 is a growth and survival factor for human myeloma cells, Blood 99 (2002) 3756-3762.
25. D. S. Mehta, A. L. Wurster, M. J. Whitters, D. A. Young, M. Collins, M. J. Grusby, IL-21 induces the apoptosis of resting and activated primary B cells, J. Immunol. 170 (2003) 4111-4118.
26. K. Ozaki, R. Spolski, C. G. Feng, C. F. Qi, J. Cheng, A. Sher, H. C. Morse, 3rd, C. Liu, P. L. Schwartzberg, W. J. Leonard, A critical role for IL-21 in regulating immunoglobulin production, Science 298 (2002) 1630-1634.
27. A. Suto, H. Nakajima, K. Hirose, K. Suzuki, S. Kagami, Y. Seto, A. Hoshimoto, Y. Saito, D. C. Foster, I. Iwamoto, Interleukin 21 prevents antigen-induced IgE production by inhibiting germ line C(epsilon) transcription of IL-4-stimulated B cells, Blood 100 (2002) 4565-4573.
28. M. Hecker, A. Bohnert, I. R. Konig, G. Bein, H. Hackstein, Novel genetic variation of human interleukin-21 receptor is associated with elevated IgE levels in females, Genes Immun. 4 (2003) 228-233.
29. F. Imani, K. E. Kehoe, Infection of human B lymphocytes with MMR vaccine induces IgE class switching, Clin. Immunol. 100 (2001) 355-361.
30. C. P. Thienes, L. De Monte, S. Monticelli, M. Busslinger, H. J. Gould, D. Vercelli, The transcription factor B cell-specific activator protein (BSAP) enhances both IL-4- and CD40-mediated activation of the human epsilon germline promoter, J. Immunol. 158 (1997) 5874-5882.
31. S. Fujieda, K. Zhang, A. Saxon, IL-4 plus CD40 monoclonal antibody induces human B cells gamma subclass-specific isotype switch: switching to gamma 1, gamma 3, and gamma 4, but not gamma 2, J. Immunol. 155 (1995) 2318-2328.
32. S. Fujieda, Y. Q. Lin, A. Saxon, K. Zhang, Multiple types of chimeric germ-line Ig heavy chain transcripts in human B cells: evidence for trans-splicing of human Ig RNA, J. Immunol. 157 (1996) 3450-3459.
33. H. H. Jabara, S. R. Brodeur, R. S. Geha, Glucocorticoids upregulate CD40 ligand expression and induce CD40L-dependent immunoglobulin isotype switching, J. Clin. Invest. 107 (2001) 371-378.
34. S. G. Tangye, A. Ferguson, D. T. Avery, C. S. Ma, and P. D. Hodgkin, Isotype switching by human B cells is division-associated and regulated by cytokines, J. Immunol. 169 (2002) 4298-4306.
35. E. M. van Leeuwen, L. E. Gamadia, P. A. Baars, E. B. Remmerswaal, I. J. ten Berge, R. A. van Lier, Proliferation requirements of cytomegalovirus-specific, effector-type human CD8+ T cells, J. Immunol. 169 (2002) 5838-5843.
36. M. Strengell, S. Matikainen, J. Siren, A. Lehtonen, D. Foster, I. Julkunen, T. Sareneva, IL-21 in synergy with IL-15 or IL-18 enhances IFN-gamma production in human NK and T cells, J. Immunol. 170 (2003) 5464-5469.
37. R. Q. Cron, CD154 transcriptional regulation in primary human CD4 T cells, Immunol. Res. 27 (2003) 185-202.
38. W. F. Rigby, M. G. Waugh, B. J. Hamilton, Characterization of RNA binding proteins associated with CD40 ligand (CD154) mRNA turnover in human T lymphocytes, J. Immunol. 163 (1999) 4199-206
39. H. Gascan, J. F. Gauchat, G. Aversa, P. Van Vlasselaer, J. E. de Vries, Anti-CD40 monoclonal antibodies or CD4+ T cell clones and IL-4 induce IgG4 and IgE switching in purified human B cells via different signaling pathways, J. Immunol. 147 (1991) 8-13.
40. M. Strengell, T. Sareneva, D. Foster, I. Julkunen, S. Matikainen, IL-21 up-regulates the expression of genes associated with innate immunity and Th1 response, J. Immunol. 169 (2002) 3600-3605.
41. N. Kobayashi, H. Nagumo, K. Agematsu, IL-10 enhances B-cell IgE synthesis by promoting differentiation into plasma cells, a process that is inhibited by CD27/CD70 interaction, Clin. Exp. Immunol. 129 (2002) 446-452.
42. S. K. Shapira, D. Vercelli, H. H. Jabara, S. M. Fu, R. S. Geha, Molecular analysis of the induction of immunoglobulin E synthesis in human B cells by interleukin 4 and engagement of CD40 antigen, J. Exp. Med. 175 (1992) 289-292.
43. A. Durandy, C. Hivroz, F. Mazerolles, C. Schiff, F. Bernard, E. Jouanguy, P. Revy, J. P. DiSanto, J. F. Gauchat, J. Y. Bonnefoy, J. L. Casanova, A. Fischer, Abnormal CD40-mediated activation pathway in B lymphocytes from patients with hyper-IgM syndrome and normal CD40 ligand expression, J. Immunol. 158 (1997) 2576-2584.
44. A. Bhushan, B. Barnhart, S. Shone, C. Song, L. R. Covey, A transcriptional defect underlies B lymphocyte dysfunction in a patient diagnosed with non-X-linked hyper-IgM syndrome, J. Immunol. 164 (2000), 2871-2880.
45. A. Jain, C. A. Ma, E. Lopez-Granados, G. Means, W. Brady, J. S. Orange, S. Liu, S. Holland, J. M. Derry, Specific NEMO mutations impair CD40-mediated c-Rel activation and B cell terminal differentiation, J. Clin. Invest. 114 (2004) 1593-1602.
46. J. F. Gauchat, H. Gascan, R. de Waal Malefyt, J. E. de Vries, Regulation of germ-line epsilon transcription and induction of epsilon switching in cloned EBV-transformed and malignant human B cell lines by cytokines and CD4+ T cells, J. Immunol. 148 (1992) 2291-2299.
47. M. Sugai, H. Gonda, T. Kusunoki, T. Katakai, Y. Yokota, A. Shimizu, Essential role of Id2 in negative regulation of IgE class switching, Nat. Immunol. 4 (2003) 25-30.
48. C. A. Akdis, T. Blesken, M. Akdis, B. Wuthrich, K. Blaser, Role of interleukin 10 in specific immunotherapy, J. Clin. Invest. 102 (1998) 98-106.
49. J. Punnonen, R. de Waal Malefyt, P. van Vlasselaer, J. F. Gauchat, J. E. de Vries, IL-10 and viral IL-10 prevent IL-4-induced IgE synthesis by inhibiting the accessory cell function of monocytes, J. Immunol. 151 (1993) 1280-1289.
50. H. Jin, R. Carrio, A. Yu, T. R. Malek, Distinct activation siganals determine whether IL-21 induces B cell costimulation, growth-arrest, or Bim-dependent apoptosis, J. Immunol. 173 (2004) 657-665.
51. S. M. Grunewald, M. Teufel, K. Erb, A. Nelde, M. Mohrs, F. Brombacher, E. B. Brocker, W. Sebald, A. Duschl, Upon prolonged allergen exposure IL-4 and IL-4Ralpha knockout mice produce specific IgE leading to anaphylaxis, Int. Arch. Allergy Immunol. 125 (2001) 322-826.
52. G. J. McKenzie, C. L. Emson, S. E. Bell, S. Anderson, P. Fallon, G. Zurawski, R. Murray, R. Grencis, A. N. McKenzie, Impaired development of Th2 cells in IL-13-deficient mice, Immunity 9 (1998) 423-432.
53. G. Grunig, M. Warnock, A. E. Wakil, R. Venkayya, F. Brombacher, D. M. Rennick, D. Sheppard, M. Mohrs, D. D. Donaldson, R. M. Locksley, D. B. Corry, Requirement for IL-13 independently of IL-4 in experimental asthma, Science 282 (1998) 2261-2263.
54. D. M. Walter, J. J. McIntire, G. Berry, A. N., D. D. Donaldson, R. H. DeKruyff, D. T. Umetsu, Critical role for IL-13 in the development of allergen-induced airway hyperreactivity. J. Immunol. 167 (2001) 4668-4675.
55. O. Hajoui, R. Janani, M. Tulic, P. Joubert, T. Ronis, Q. Hamid, H. Zheng, Synthesis of IL-13 by human B lymphocytes: Regulation and role in IgE production, J. Allergy Clin. Immunol. 114 (2004) 657-663.
56. T. A. Platts-Mills, T. A, The role of immunoglobulin E in allergy and asthma, Am. J. Respir. Crit. Care Med. 164 (2001) S1-S5.
57. J. Pene, J.-F. Gauchat, S. Lecart, E. Drouet, P. Gugliemi, V. Boulay, A. Delwail, D. Foster, J.-C. Lecron, H. Yssel, IL-21 is a switch factor for the production of IgG1 and IgG3 by human B cells, J. Immunol. 172 (2004) 5154-5157.
58. J. M. Purkerson, P. C. Isakson, Independent regulation of DNA recombination and immunoglobulin (Ig) secretion during isotype switching to IgG1 and IgE, J. Exp. Med. 179 (1994) 1877-1883.
59. C. Ninomiya, H. L. Spiegelberg, IL-4 and transforming growth factor-beta suppress human immunoglobulin secretion in vitro by surface IgD-B cells, Clin. Exp. Immunol. 89 (1992) 261-268.
60. B. A. de Boer, Y. C. Kruize, M. Yazdanbakhsh, In vitro production of IgG4 by peripheral blood mononuclear cells (PBMC): the contribution of committed B cells, Clin. Exp. Immunol. 114 (1998) 252-257.
61. Mehta D. S., A. L. Wurster, M. J. Grusby, Biology of IL-21 and the IL-21 receptor. Immunol Rev 202 (2004) 84-95.
62. K. Ozaki, R. Spolski, R. Ettinger, H. P. Kim, G. Wang, C. F. Qi, P. Hwu, D. J. Shaffer, S. Akilesh, D. C. Roopenian, H. C. Morse, 3^{rd}, P. E. Lipsky, W. J. Leonard, Regulation of B cell differentiation and plasma cell generation by IL-21, a novel inducer of Blimp-1 and Bcl-6, J. Immunol. 173 (2004) 5361-5371.

Those skilled in the art will recognize, or be able to ascertain using no more than routine experimentation, many equivalents of the specific embodiments of the invention described herein. Such equivalents are intended to be encompassed by the following claims.

What is claimed is:

1. A method of ameliorating a symptom of an atopic disorder in a human subject, the method comprising:
administering, to the subject, a human IL-21 polypeptide that comprises the amino acid sequence of SEQ ID NO:2, in an amount effective for ameliorating at least one symptom of the atopic disorder.

2. The method of claim 1 wherein the atopic disorder is selected from the group consisting of: atopic dermatitis, asthma, extrinsic bronchial asthma, urticaria, eczema, allergic rhinitis, and allergic enterogastritis.

3. The method of claim 1 wherein IgE levels are decreased by at least 40% relative to levels in the subject prior to the administering.

4. The method of claim 1 further comprising evaluating one or more symptoms of the atopic disorder in the subject.

5. The method of claim 1 further comprising evaluating an IL-21 associated parameter in the subject.

6. The method of claim 1 further comprising evaluating levels of endogenous IgE in the subject.

7. A method of treating or preventing an atopic disorder in a human subject, the method comprising:
administering, to the subject, a human IL-21 polypeptide that comprises the amino acid sequence of SEQ ID NO:2, in an amount effective for treating or preventing the atopic disorder.

8. The method of claim 7 wherein the atopic disorder is selected from the group consisting of: atopic dermatitis, asthma, extrinsic bronchial asthma, urticaria, eczema, allergic rhinitis, and allergic enterogastritis.

9. A method of modulating IgG and/or IgE production in a cell, the method comprising:
contacting an IL-21 pathway agonist that is an IL-21 polypeptide or a nucleic acid encoding an IL-21 polypeptide, to the cell in an amount sufficient to increase IgG production and/or decrease IgE production.

10. The method of claim 9 wherein the IL-21 pathway agonist is an IL-21 polypeptide.

11. The method of claim 9 wherein IgE levels are decreased by at least 40%.

12. The method of claim 9 wherein the cell is in vitro.

13. The method of claim 9 wherein the cell is in vivo.

14. The method of claim 9 wherein IgE/IgG ratio is decreased.

15. A method of modulating IgG and/or IgE production in a cell, the method comprising:
contacting an IL-21 pathway antagonist, to the cell in an amount sufficient to decrease IgG and/or increase IgE production, wherein the antagonist is selected from the group consisting of: (i) an antibody that binds IL-21, (ii) a polypeptide that comprises a soluble form of the IL-21 receptor, or (iii) a nucleic acid that reduces expression of IL-21, IL-21 receptor, or an IL-21 pathway component.

16. The method of claim 15 wherein IgE levels are increased by at least 20%.

17. The method of claim 15 wherein IgE/IgG ratio is increased.

18. The method of claim 17 wherein the ratio is increased by at least 20%.

19. A pharmaceutical composition comprising a human IL-21 polypeptide and a second agent for treating an atopic disorder.

20. A method of evaluating a subject having or suspected of having an atopic disorder, the method comprising
evaluating an IL-21 associated parameter for a subject having an atopic disorder
comparing results of the evaluating to a reference parameter, and
providing a recommendation of a therapy for the disorder as a function of the comparison.

21. The method of claim 20 wherein the IL-21 associated parameter comprises a quantitative or qualitative value for IL-21 polypeptide abundance or IL-21 mRNA.

22. The method of claim 20 wherein the IL-21 associated parameter comprises a quantitative or qualitative value for IL-21 receptor protein or mRNA, or for an IL-21 pathway activity.

23. The method of claim 20 wherein the atopic disorder is selected from the group consisting of: atopic dermatitis, asthma, extrinsic bronchial asthma, urticaria, eczema, allergic rhinitis, and allergic enterogastritis.
